(19) United States
(12) Patent Application Publication
(10) Pub. No.: US 2023/0090140 A1
(43) Pub. Date: Mar. 23, 2023

(54) Catheter Tray, Packaging System, Instructional Inserts, and Associated Methods

(71) Applicant: Medline Industries, LP, Northfield, IL (US)

(72) Inventor: Lucas Crosby, Crystal Lake, IL (US)

(21) Appl. No.: 17/685,064

(22) Filed: Mar. 2, 2022

Related U.S. Application Data
(60) Provisional application No. 63/247,733, filed on Sep. 23, 2021.

(51) Int. Cl. A61M 25/00

(52) U.S. Cl. CPC A61M 25/002; A61M 25/0017

(57) ABSTRACT

A tray (100) for accommodating a coiled medical device, such as a catheter assembly (700), includes a first compartment (101), a second compartment (102), and a third compartment (103). The catheter assembly (700) and devices associated with a catheterization procedure, such as syringes (706,707) containing sterile water and lubricating jelly and a specimen container (708) can be disposed within the tray. Instructional banners (1600,1700,1800,1900,2000,2100, 2200,2300) can be included with the tray (100). One or more layers of wrap material (2200) can be folded about the tray (100) to enclose the tray (100) and other items, such as an additional layer of wrap material (800), packaged liquid skin sanitizer (709), an instructional banner (1600), and packaged gloves (712).

### CROSS REFERENCE TO PRIOR APPLICATIONS

This application claims priority and benefit under 35 USC §119(e) from US Provisional Application Ser. No. 63/247,733, filed Sep. 23, 2021, which is incorporated by reference for all purposes.

### BACKGROUND

### Technical Field

This disclosure relates generally to medical kits, and more particularly to medical kits for

Foley catheters, related medical devices, as well as an instruction manual included therewith.

### Background Art

Medical devices, including surgical instruments, supplies, and so forth, are generally shipped from manufacturer to medical services provider in sterile packaging. For example, a scalpel may be shipped to a surgeon in a plastic, vacuum-sealed, sterile package. Similarly, bandages may be shipped in paper, plastic, or paper composite sterile wrappers. When the medical services provider is ready to use the medical supply, the sterile package is removed. The medical services provider then uses the object in accordance with the procedure being performed.

While conventional packaging works well for objects having a generally unchanging form factor, special considerations have to be taken into consideration for some medical supplies. By way of example, catheter assemblies and other flexible equipment is generally shipped in a coiled configuration. Once the sterile packaging is removed, the catheter must be uncoiled prior to use. Care must be taken in shipping, unwrapping, and using the catheter. For instance, if a catheter is inadvertently bent, kinked, or otherwise damaged, it may no longer be suitable for use.

Compounding this issue, catheters are available in a variety of lengths ranging from 100 centimeters to over 250 centimeters.

Traditional catheters are packaged, for example, in individual packaging. The catheter and card are then sealed in a sterile plastic wrap. These catheters are prone to damage in shipment, storage, and when being unpacked, as the card and wrap provide little physical protection.

Some manufacturers have started shipping catheters and other similar devices in flat plastic trays. For example, U.S. Pat. No. 6,068,121 to McGlinch teaches one such tray. The tray has several specifically contoured loops such that one universal tray will accommodate several different sized catheters. Such packaging presents a problem, however, in that large amounts of storage space are taken with a universal tray, especially when a relatively short catheter is shipped therein. Additionally, when in use, these trays occupy large amounts of a medical service provider's sterile workspace or table, leaving little room for related components, such as lubricants, fluid bags, and so forth.

There is thus a need for an improved container for flexible medical devices or catheters that facilitates more effective and simpler deployment of the device during a procedure.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates a top, front, right perspective view of one embodiment of a tray for a catheter or similar assembly in accordance with embodiments of the disclosure.

FIG. 2 illustrates a top, front, left perspective view of one embodiment of a tray for a catheter or similar assembly in accordance with embodiments of the disclosure.

FIG. 3 illustrates a top plan view of one embodiment of a tray for a catheter or similar assembly in accordance with embodiments of the disclosure.

FIG. 4 illustrates a front elevation view of one embodiment of a tray for a catheter or similar assembly in accordance with embodiments of the disclosure.

FIG. 5 illustrates a cut-away, left elevation view of one embodiment of a tray for a catheter or similar assembly in accordance with embodiments of the disclosure.

FIG. 6 illustrates a bottom plan view of one embodiment of a tray for a catheter or similar assembly in accordance with embodiments of the disclosure.

FIG. 7 illustrates a top plan view one explanatory Foley catheter, coiled tubing, and fluid drainage bag.

FIG. 8 illustrates one explanatory medical kit, as well as one or more explanatory steps for manufacturing the medical kit, in accordance with one or more embodiments of the disclosure.

FIG. 9 illustrates one explanatory medical kit, as well as one or more explanatory steps for manufacturing the medical kit, in accordance with one or more embodiments of the disclosure.

FIG. 10 illustrates one explanatory medical kit, as well as one or more explanatory steps for manufacturing the medical kit, in accordance with one or more embodiments of the disclosure.

FIG. 11 illustrates one explanatory medical kit, as well as one or more explanatory steps for manufacturing the medical kit, in accordance with one or more embodiments of the disclosure.

FIG. 12 illustrates one explanatory medical kit, as well as one or more explanatory steps for manufacturing the medical kit, in accordance with one or more embodiments of the disclosure.

FIG. 13 illustrates one explanatory medical kit, as well as one or more explanatory steps for manufacturing the medical kit, in accordance with one or more embodiments of the disclosure.

FIG. 14 illustrates one explanatory medical kit, as well as one or more explanatory steps for manufacturing the medical kit, in accordance with one or more embodiments of the disclosure.

FIG. 15 illustrates one explanatory medical kit in accordance with one or more embodiments of the disclosure.

FIG. 16 illustrates one explanatory instructional banner in accordance with one or more embodiments of the disclosure.

FIG. 17 illustrates another explanatory instructional banner in accordance with one or more embodiments of the disclosure.

FIG. 18 illustrates another explanatory instructional banner in accordance with one or more embodiments of the disclosure.

FIG. 19 illustrates still another explanatory instructional banner in accordance with one or more embodiments of the disclosure.

FIG. 20 illustrates yet another explanatory instructional banner in accordance with one or more embodiments of the disclosure.

FIG. 21 illustrates another explanatory instructional banner in accordance with one or more embodiments of the disclosure.

FIG. 22 illustrates one explanatory syringe with an instructional banner in accordance with one or more embodiments of the disclosure.

FIG. 23 illustrates one explanatory syringe with an instructional banner in accordance with one or more embodiments of the disclosure.

FIG. 24 illustrates one explanatory method in accordance with one or more embodiments of the disclosure.

FIG. 25 illustrates one explanatory tray with an instructional banner in accordance with one or more embodiments of the disclosure.

FIG. 26 illustrates one explanatory medical kit in accordance with one or more embodiments of the disclosure.

FIG. 27 illustrates one explanatory medical kit in accordance with one or more embodiments of the disclosure.

FIG. 28 illustrates one explanatory medical kit in accordance with one or more embodiments of the disclosure.

FIG. 29 illustrates one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE EMBODIMENTS

Before describing in detail embodiments that are in accordance with the present disclosure, it should be observed that the embodiments reside primarily in combinations of method steps and apparatus components related to packaging Foley catheters, their corresponding coiled tubing, fluid drain bags, and other components in a tray. Alternate implementations are included, and it will be clear that any method steps may be executed out of order from that shown or discussed, including substantially concurrently or in reverse order, depending on the desired application. Accordingly, the apparatus components and method steps have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, it is expected that one of ordinary skill, notwithstanding possibly significant effort and many design choices motivated by, for example, available time, current technology, and economic considerations, when guided by the concepts and principles disclosed herein will be readily capable of generating such assemblies and executing such method steps with minimal experimentation.

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions.

As used herein, components may be “operatively coupled” when information can be sent between such components, even though there may be one or more intermediate or intervening components between, or along the connection path. The terms “substantially,” “essentially,” “approximately,” “about,” or any other version thereof, are defined as being close to as understood by one of ordinary skill in the art, and in one non-limiting embodiment the term is defined to be within ten percent, in another embodiment within five percent, in another embodiment within one percent and in another embodiment within one-half percent. The term “coupled” as used herein is defined as connected, although not necessarily directly and not necessarily mechanically. Also, reference designators shown herein in parenthesis indicate components shown in a figure other than the one in discussion. For example, talking about a device (10) while discussing figure A would refer to an element, 10, shown in figure other than figure A.

Embodiments of the disclosure provide a medical procedure kit comprising a tray. In one or more embodiments, the tray comprises a first compartment and a second compartment. The first compartment is at least partially bounded by a first base member in one or more embodiments. The second compartment is at least partially bounded by a second base member in one or more embodiments.

In one or more embodiments, at least one syringe is situated within the first compartment.

In one or more embodiments, a Foley catheter is situated in the second compartment. In one or more embodiments, the Foley catheter is operatively coupled to coiled tubing. In one or more embodiments, the coiled tubing is operatively coupled to a fluid drain bag. In one or more embodiments where the Foley catheter is disposed in the second compartment, the operatively coupled coiled tubing and fluid drain bag are also disposed within the second compartment. In other embodiments, such as when a medical kit is configured as a two-layer tray, the Foley catheter can be placed in the top tray with at least some of the coiled tubing, while the remainder of the coiled tubing and the fluid drain bag can be placed in the lower tray.

Embodiments of the present disclosure further provide a medical procedure kit that includes medical products for performing a medical procedure. In one embodiment, the medical procedure kit is configured for a Foley catheterization procedure. Such an embodiment will be used herein for illustration purposes. However, it will be clear to those of ordinary skill in the art having the benefit of this disclosure that embodiments of the disclosure are not so limited. Other medical procedure kits for performing other procedures could be substituted for the illustrative Foley catheterization procedure kit disclosed herein by substituting other medical implements for the Foley catheterization implements.

When a Foley catheter assembly is inserted into a patient, sterile water may be used to inflate the balloon of the Foley catheter. Additionally, the Foley catheter may be coated in a lubricating jelly prior to insertion into the patient. Fluids and other samples may then be monitored and obtained from the patient as they pass into the Foley catheter, through the coiled tubing (which may be uncoiled at this point) and into a syringe via a Luer access port or into the fluid drain bag. Embodiments of the present disclosure provide a tray configured to accommodate not only the Foley catheter, the operatively coupled coiled tubing, and the operatively coupled fluid bag, but also syringes containing fluids such as sterile water or aqueous lubricants. Further, the tray can accommodate a sterile specimen jar for capturing samples taken from the patient as they pass into the Foley catheter, through the coiled tubing (which may be uncoiled at this point) and into a syringe via a Luer access port or into the fluid drain bag.

In addition to simply accommodating these corresponding medical devices, in one or more embodiments the tray is configured to provide the medical services provider with mnemonic devices instructing them in which order to use each device. For example, a compartment containing syringes, in one embodiment, includes a base member that presents each syringe at, for example, an easy to reach angle and/or at different heights based upon order in which each syringe is used in a Foley catheterization procedure.

Another advantage of embodiments of the present disclosure is that compartments have multi-purpose functionality. For example, in one embodiment, a compartment configured to accommodate a syringe having lubricating jelly disposed therein is also configured for use as a lubricating jelly applicator or lubricating jelly application compartment. In one or more embodiments, a medical services provider first dispenses the lubricating jelly into the compartment from which the syringe was taken. The medical services provider then passes at least a tip of the Foley catheter from the second compartment into the first compartment and into the lubricating jelly. Advantageously, the tray not only serves as a shipping and storage container for an assembly of devices used with a Foley catheter procedure, but also as an application device to assist a medical services provider in using those products together, and without compromising the sterile field required for proper aseptic technique.

Embodiments of the disclosure provide a medical procedure kit that can include at least one tray. The kit can include only a single tray in some embodiments. In other embodiments, the kit includes multiple trays stacked a two-layered configuration. Trays configured in accordance with one or more embodiments of the disclosure can include one or more instructional banners. The instructional banners can be attached to the tray, attached to wrap layers positioned around the tray, or tucked within folds of the wrap layers positioned around the tray. The instructional information can inform medical personnel how to apply a Foley catheter to a patient. The instructional information positioned on the instructional banners can also inform medical personnel—or patients themselves—how to care for, clean, and properly maintain a Foley catheter when applied to a patient as well.

Other embodiments will be described below. Variants will be obvious to those of ordinary skill in the art having the benefit of this disclosure.

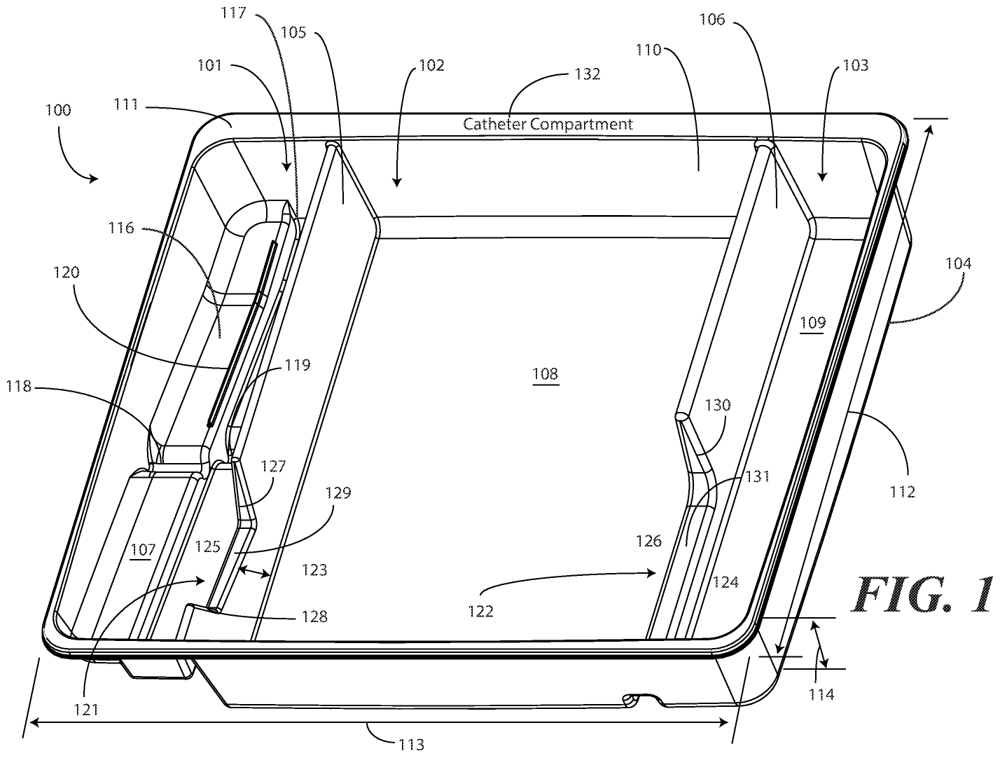

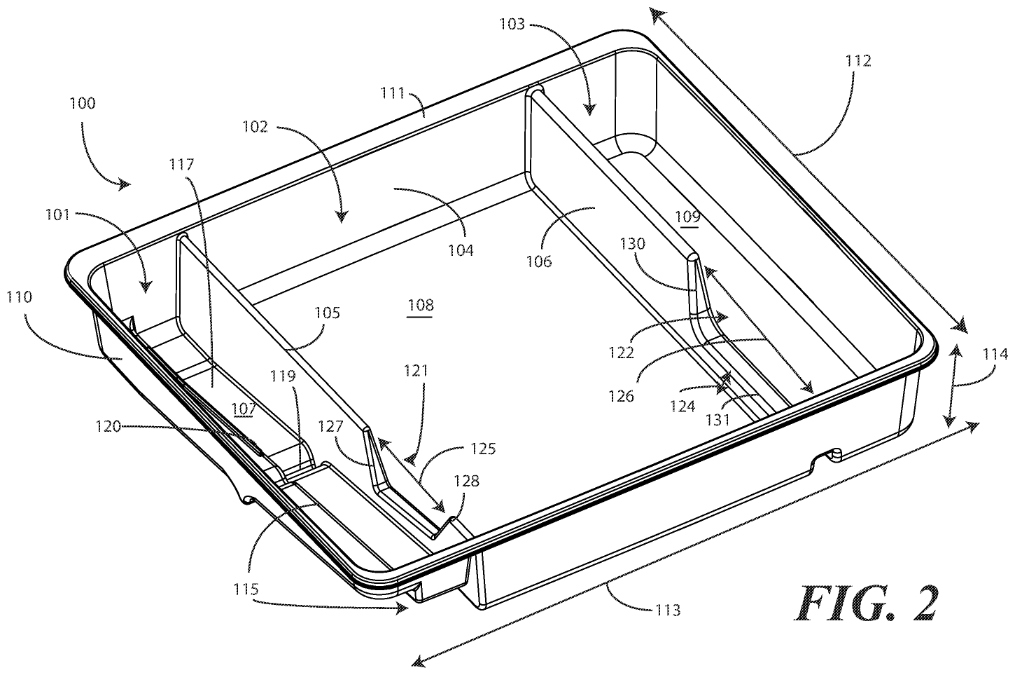

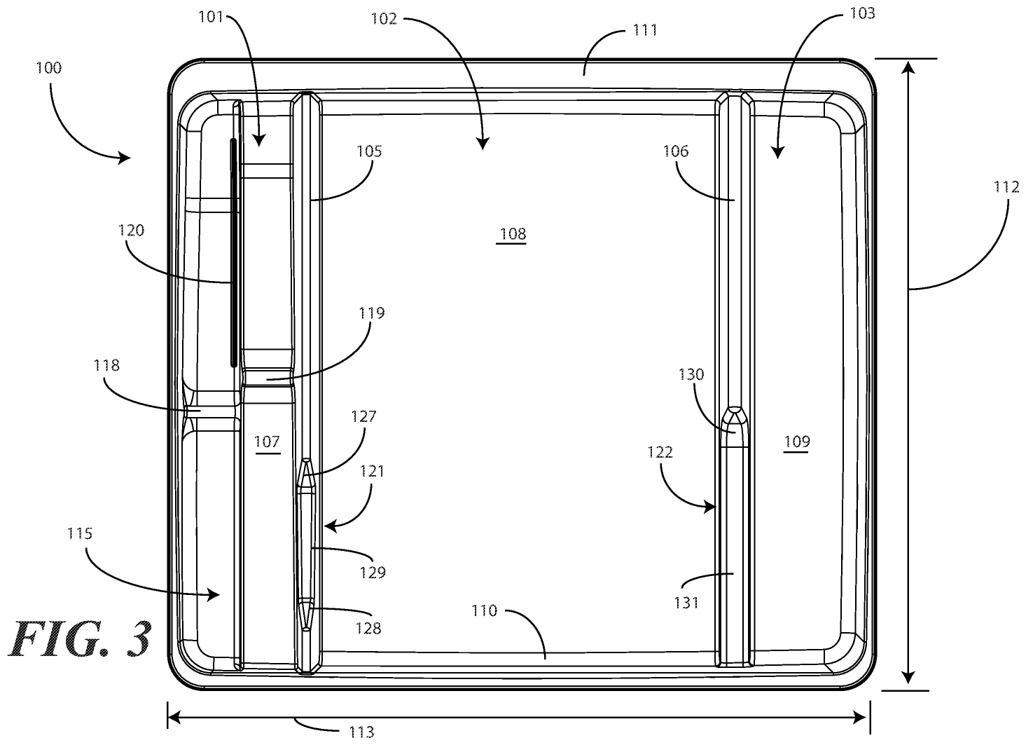

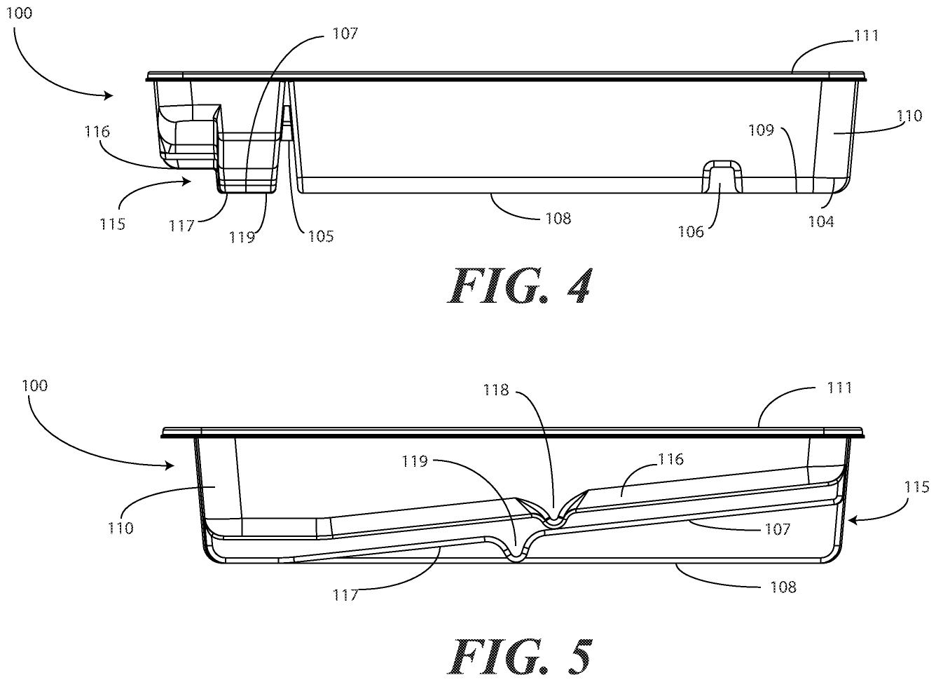

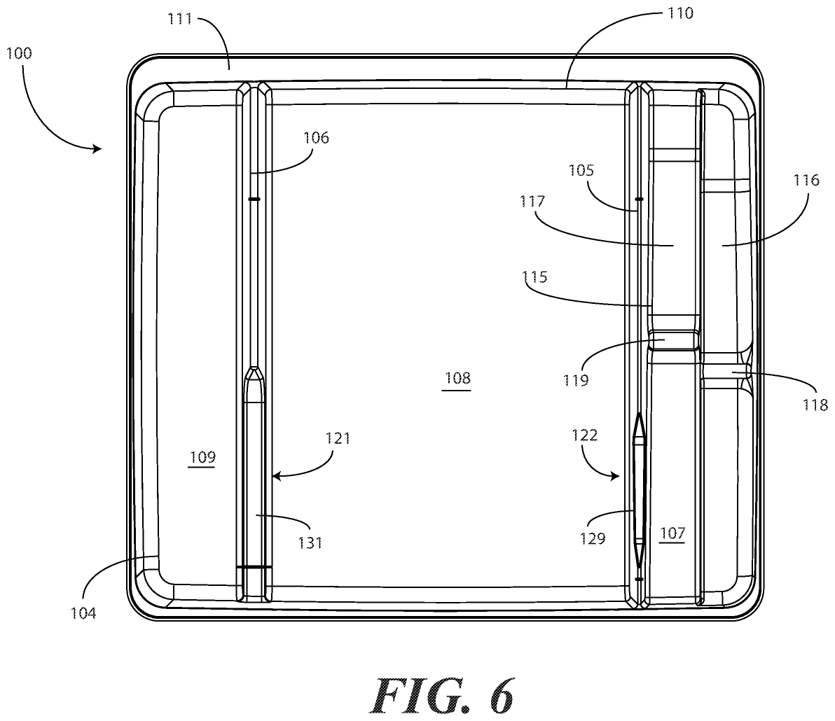

Turning now to FIGS. 1-6, illustrated therein are views of one embodiment of a tray 100 configured in accordance with one or more embodiments of the disclosure to accommodate a Foley catheter, its operatively coupled coiled tubing, and its operatively coupled fluid drain bag. FIG. 1 illustrates a top, front right perspective view of the tray 100. FIG. 2 illustrates a top, front, left perspective view of the tray 100. FIG. 3 illustrates a top plan view of the tray 100. FIG. 4 illustrates a front elevation view of the tray 100. FIG. 5 illustrates a cut-away, left elevation view of the tray 100. Likewise, FIG. 6 illustrates a bottom plan view of the tray 100. For simplicity of discussion, these figures will be referred to collectively with like reference numerals referring to identical or functionally similar elements throughout the separate views.

The tray 100 is formed by a contoured surface 104 that defines the various features and compartments of the tray 100. The contoured surface 104 of the tray 100 can be manufactured in various ways. For example, the tray 100 can be thermally formed on a mold from a soft thermoplastic, such as styrene or polystyrene. The tray 100 can be injection molded. The tray can be poured on a mold using a quick setting plastic, epoxy, or resin.

Explanatory dimensions of the tray 100 are as follows: The length 112 can be between nine and twelve inches, such as ten inches. One illustrative length 112 may be 10.380 inches. Similarly, the width 113 can be between eight and eleven inches, such as nine inches. One illustrative width 113 is 9.250 inches. The height 114 can be between one and three inches. One illustrative height 114 is 1.750 inches.

The tray 100 includes three main compartments: a first compartment 101, a second compartment 102, and a third compartment 103. The first compartment 101 is separated from the second compartment 102 by a first barrier 105. The second compartment 102 is separated from the third compartment 103 by a second barrier 106.

The compartments are open from the top of the tray 100—the top being opposite the base members of the tray 100—and are bounded on the bottom by a first compartment base member 107, a second compartment base member 108, and a third compartment base member 109. The compartments are bounded on the sides by a perimeter wall 110. The perimeter wall 110 ends in a horizontal flange 111 extending substantially orthogonally from the perimeter wall 110. The top of the tray 100 could have a hinged or snap-coupled lid that is opened or removed to reveal the compartments there beneath. Similarly, the horizontal flange 111 can be omitted, with each of the first compartment 101, the second compartment 102, and the third compartment 103 being bounded by a wall, be it the perimeter wall 110 or another wall, that extends distally from the various compartment base members 107,108,109 to a terminating edge.

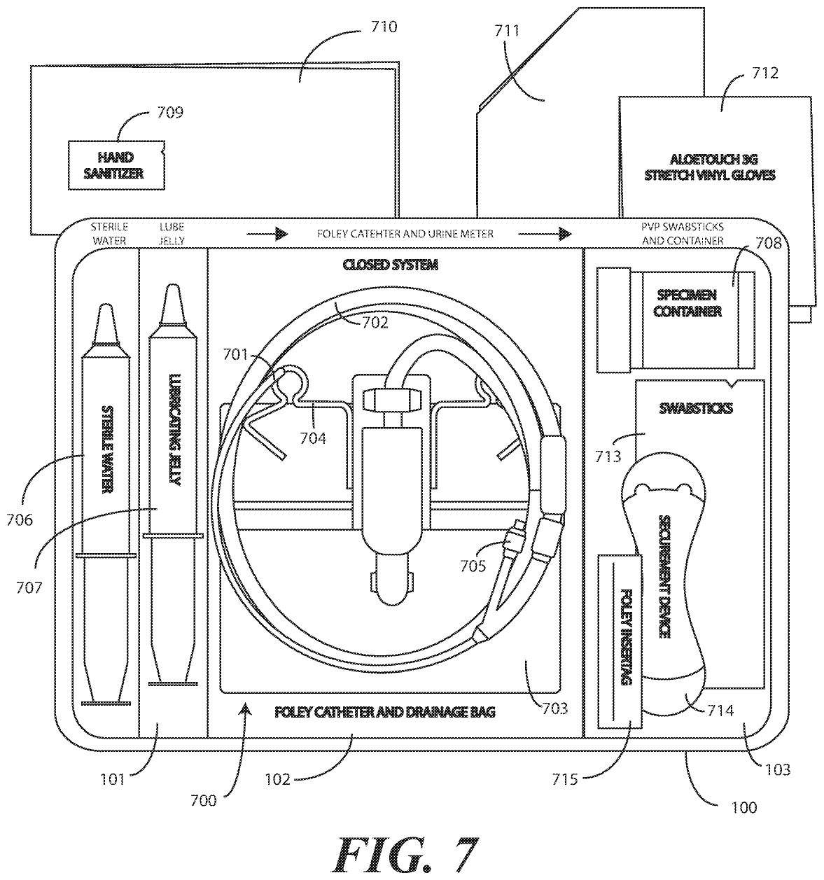

The tray 100 is configured to hold or otherwise accommodate the necessary devices and materials to perform a Foley catheter-based procedure on a patient. The tray 100 is configured to hold not only the Foley catheter, its operatively coupled coiled tubing, and its operatively coupled fluid drain bag, but the medical devices corresponding to Foley catheter use as well. Using one illustrative procedure as an example, the following devices will be used: a syringe holding sterile water, a syringe holding lubricating jelly or another equivalent lubricant, a catheter assembly, skin cleansing or preparation materials, and a specimen jar. The various compartments and features of the tray 100 shown in FIGS. 1-6 will be described for use with these devices. As will be described in more detail below with reference to FIG. 7, additional objects can be included with the tray, such as one or more towels, a drape to cover the patient, rubber gloves, hand sanitizing materials, swab sticks, a securement device, a Foley insert tag, a printed instruction pamphlet, and so forth.

For procedures using the syringe holding sterile water, syringe holding lubricating jelly, the Foley catheter, its operatively coupled coiled tubing, and its operatively coupled fluid drain bag, and specimen jar, the tray 100 is configured such that these objects are ordered in accordance with their order of use during the Foley catheterization procedure. For example, the tray 100 includes a first compartment 101 for accommodating one or more syringes, a second compartment 102 for accommodating the Foley catheter, its operatively coupled coiled tubing, and its operatively coupled fluid drain bag, and optionally one or more syringes, and a third compartment 103 for accommodating at least the specimen jar. These devices stowed in the various compartments will be illustrated and described with respect to FIG. 7 below. The discussion of FIGS. 1-6 will include the features of the tray 100 that make the tray 100 suitable for accommodating these devices.

The first compartment base member 107 is contoured. The first compartment base member 107 can include features that hold any syringes stored within the first compartment 101 at different heights. The first compartment base member 107 includes a stair-stepped contour 115 suitable that supports syringes at different heights.

A first step portion 116 of the stair-stepped contour 115 may be at a different height within the tray 100 than a second step portion 117 of the stair-stepped contour 115. The first step portion 116—which is disposed farther from the first barrier 105 than the second step portion 117—is shallower than the second step portion 117. Said differently, the second step portion 117 is disposed at a greater depth within the tray 100 than the first step portion 116.

The contours of the first compartment 101 can serve as mnemonic device when for medical implements stored within the first compartment 101. For example, it may be intuitive that a higher syringe may need to be used first. Accordingly, placing this “first to be used” syringe on a higher step portion will indicate that this syringe should to be used first. This intuition is further enforced when the higher position is situated farther to the left in a left-to-right usage, i.e., like reading a book, configuration. Thus, a user receives a mnemonic reminder to use a syringe disposed on the first step portion 116 prior to a syringe disposed on the second step portion 117, as it is both higher and farther to the left.

Where syringes are stowed in the first compartment 101, the first compartment base member 107 can further be configured for syringe ease of use. For example, the first compartment base member 107 is inclined relative to other compartment base members. In FIGS. 1-6, the second compartment base member 108 and third compartment base member 109 are substantially coplanar with each other. Further, the second compartment base member 108 and third compartment base member 109 are generally flat in these views. The first compartment base member 107 could be substantially flat.

In this illustrative tray 100, the first compartment base member 107 is configured to be inclined relative to one or both of the second compartment base member 108 and third compartment base member 109. As such, the stair-stepped contour 115 forms a ramp upon which syringes may be placed so that the plunger of each syringe is predisposed to project upward and out of the tray 100. Said differently, the stair-stepped contour 115 is configured such that the first step portion 116 and the second step portion 117 are disposed in a non-parallel orientation relative to the second compartment base member 108. This configuration makes it easier for a medical services provider to grasp the syringes and remove them from the tray 100.

The first compartment base member 107 may include other features suitable for accommodating one or more syringes as well. Any of the first compartment base member 107, the first step portion 116, or the second step portion 117 can include recesses 118,119 for accommodating a syringe flange. These recesses 118,119 generally function to prevent the syringes from sliding lengthwise within the first compartment 101. Similarly, any of the first compartment base member 107, the first compartment sidewalls, the first step portion 116, or the second step portion 117 include protrusions 120 that help to prevent the syringes from sliding laterally within the first compartment 101.

One or both of the first barrier 105 and the second barrier 106 include optional openings disposed therein. In FIGS. 1-6, the first barrier 105 includes a first opening 121 between the first compartment 101 and the second compartment 102. Similarly, the second barrier 106 includes a second opening 122 between the second compartment 102 and the third compartment 103. Each of these openings has an opening depth associated therewith. Similarly, each opening has an opening width associated therewith. In FIGS. 1-6, the first opening 121 is bounded by a first opening base member 129 and two inclined first opening side members 127,128, while the second opening 122 is bounded by a second opening base member 131, an inclined second opening side member 130, and the perimeter wall 110.

While the opening depths can be the same, the opening depths are different in FIGS. 1-6.

For example, in FIGS. 1-6, the first opening 121 has a first opening depth 123 that is less than the second opening depth 124 of the second opening 122. Similarly, the opening widths are different. For example, in FIGS. 1-6, the first opening 121 has a first opening width 125 that is less than the second opening width 126 of the second opening 122. Such a disparity in opening depths and widths, as well as the inclusion of inclined opening side members, provides an advantage in some applications.

For instance, in many Foley catheter procedures a pair of syringes—such as syringes having a one-half inch diameter—fits easily into the first compartment 101 when the tray 100 is made with the illustrative dimensions set forth above. However, some procedures require one or more of the syringes to be larger. Additionally, in still other procedures syringes are used in different orders. Where syringes that are larger in diameter are required, these larger syringes are capable of nesting within the first opening 121 and second opening 122. The inclined opening side members prevent the syringe from moving lengthwise, while the disparate opening heights present the plunger of the syringe to the medical services provider for easy removal from the tray 100. Where the syringes are used in different orders, or simply for design choice, one syringe can be placed in the first compartment 101, while another syringe can be placed in the second compartment 102, and so forth.

The stair-stepped contour 115, working in tandem with the first opening 121, gives the tray additional advantages. For instance, the first compartment base member 107 allows the first compartment 101 to be used as a lubricant applicator for the catheter.

Specifically, the medical services provider may dispense the lubricating jelly into the first compartment 101 after removing the syringes therefrom. This lubricating jelly is dispensed along the second step portion 117. Where included, and where lower than the first step portion 116, the second step portion 117 serves as a channel in which the lubricating jelly may spread. A medical services provider may then pass the at least a tip of the Foley catheter from the first compartment 101 into the second compartment 102, optionally through the first opening 121, and into the lubricating jelly. The tip or more of the Foley catheter passes through the channel formed by the second step portion 117, i.e., along the second step portion 117 through the dispensed lubricating jelly. From there, the Foley catheter is passed out the top of the tray 100 to the patient.

This feature of the tray 100 greatly eases the application of lubricating jelly to the catheter. The tray 100 is packaged with printed instructions showing the medical services provider how to apply lubricating jelly in this manner.

Alternative methods may be used to apply the lubricating jelly as well. For example, the lubricating jelly can be dispensed directly onto the Foley catheter and/or its operatively coupled tubing while these components are situated in or above either the first compartment 101 or the second compartment 102. Excess lubricant falling from the catheter tubing can then collect, and be retained, in any of the first compartment 101, the second step portion 117 of the first compartment 101, or the second compartment 102.

This particular feature highlights another advantage of the “compartmentalized” structure. As the tray 100 includes multiple compartments, various tasks associated with a Foley catheterization procedure can be completed while keeping the Foley catheter within the tray 100. The ability to keep the catheter in the tray 100 reduces the risk that the Foley catheter, its operatively coupled coiled tubing, and its operatively coupled fluid drain bag, or corresponding devices, will be contaminated with bacteria or microbes on other objects within the procedure room.

Illustrating by example, when the first compartment 101 is used to apply lubricating jelly to the Foley catheter or its operatively coupled coiled tubing, the lubricating jelly can be applied while these components are completely contained within the confines of the tray 100. This reduces the risk that the Foley catheter or its operatively coupled coiled tubing will become contaminated. This correspondingly reduces the risk of infection for the patient receiving the catheter.

Systems in which the catheterization procedure components are shipped in separate and/or stacked containers may contribute to substandard techniques in that the catheter can become contaminated when moving it from its shipping container. Consequently, the patient can be at an elevated risk of infection as the catheter is moved from one tray to another. A solution to this problem is the provision of a tray 100 with compartments. Further, the first compartment 101 allows the Foley catheter can stay in place during and after lubrication. By having easy access to the components disposed in the tray 100, the medical services provider can more easily prepare and use the components within the tray 100. This helps to minimize the risk of contaminating the patient or the sterile field during the procedure.

The second step portion 117 is configured to be inclined at a shallower angle than the first step portion 116 in at least a portion opposite the recess 119 from the first opening 121. When configured in such a fashion, the second step portion 117 includes a “cutdown” so that the catheter can stay within the channel both during and after lubrication.

Additionally, the Foley catheter can be placed in both the first opening 121 and second opening 122 during lubrication. When positioned in this configuration, the second opening 122 helps to align the catheter with the first opening for easy passage through the lubrication channel formed by the second step portion 117.

The tray 100 of FIGS. 1-6 includes additional advantages. For example, instructions 132 or other graphical indicia can be printed, placed upon, or molded into the tray. Moreover, instructional banners can be attached to the tray, attached to wrap layers positioned around the tray, or tucked within folds of the wrap layers positioned around the tray. The instructional information can inform medical personnel how to apply a Foley catheter to a patient. The instructional information positioned on the instructional banners can also inform medical personnel—or patients themselves—how to care for, clean, and properly maintain a Foley catheter when applied to a patient as well.

In one or more embodiments, the graphical indicia are molded into the horizontal flange 111. However, it can be molded into other portions of the tray 100, including the base members of the compartments, along the barriers, and so forth. Compartment designations can be placed above each compartment to ensure the medical services provider uses the correct device or material at the correct time. Expiratory dates for materials or devices disposed within the tray 100 may be molded into portions of the tray 100.

Another advantage of the tray 100 is that its compartmentalized configuration helps to reduce the risk of contaminating a patient or compromising the sterile nature of the components stored in the tray 100. Since a Foley catheter, its operatively coupled coiled tubing, and its operatively coupled fluid drain bag, and medical devices corresponding to Foley catheter use, are stored within the same tray 100, the risk of cross-contamination between sterile work areas and non-sterile spaces is minimized Further, by having the Foley catheter, its operatively coupled coiled tubing, and its operatively coupled fluid drain bag, and the devices corresponding to catheter use stowed in a one-level tray rather than a multi-level, stacked configuration, the medical services provider can more easily prepare and use the catheter and corresponding devices disposed within the tray 100.

Turning now to FIG. 7, illustrated therein is one explanatory embodiment of a medical kit configured in accordance with one or more embodiments of the disclosure. A catheter assembly comprises a Foley catheter 701, its operatively coupled coiled tubing 702, and its operatively coupled fluid drain bag 703. As shown in FIG. 7, the coiled tubing 702 is coupled between the Foley catheter 701 and the fluid drain bag 703.

A clip or other hanger 704 can be coupled to the fluid drain bag 703 so that the fluid drain bag 703 can be coupled to a stand, bedrail, or other object. The coiled tubing 702 may be coupled to the Foley catheter 701, and the fluid drain bag 703 coupled to the coiled tubing 702, before the assembly is placed in the tray 100. Alternatively, the Foley catheter 701, the coiled tubing 702, and the fluid drain bag 703 can be separately stored in a tray, with each being coupled together once the tray is opened.

The Foley catheter 701 includes a drainage port 705, which is operatively coupled to the coiled tubing 702. The Foley catheter 701 can optionally include an inflation port. A syringe containing sterile water or other fluid can be coupled to the inflation port. The sterile water or other fluid can be injected into the inflation port so that the balloon of the Foley catheter 701 can be inflated.

The Foley catheter 701 is suitable for insertion into the urethra of a patient. The Foley catheter 701 is an elongated, flexible device. The Foley catheter 701 can be manufactured from flexible material such as polymers, elastomers, flexible steel, or other materials. Other materials suitable for manufacturing the Foley catheter 701 will be obvious to those of ordinary skill in the art.

The Foley catheter 701 includes an insertion end and a second end, which is coupled to the coiled tubing 702. A central lumen passes through the Foley catheter 701 so that fluids may pass from the insertion end to the second end, through the operatively coupled coiled tubing 702, and into the fluid drain bag 703.

When inserted into a patient, an inflatable retaining balloon (not shown) operatively coupled to the Foley catheter 701 can be inflated using the inflation port to retain the Foley catheter 701 within the patient. The balloon can be inflated with sterile water to retain the insertion end of the Foley catheter 701 within the patient's bladder.

FIG. 7 illustrated the tray 100 of FIGS. 1-6 having the catheter assembly 700, syringes 706,707, and a specimen container 708 stored therein as a catheter packaging system. The illustrative catheter packaging system of FIG. 7 includes a tray 100 having a first compartment 101, a second compartment 102, and a third compartment 103.

The first compartment 101 is configured to accommodate syringes 706,707. The second compartment 102 is configured to accommodate a coiled medical device, such as catheter assembly 700. The third compartment 103 is configured to accommodate the specimen container 708. The third compartment 103 can accommodate other materials as well, including skin sanitizers 709 and cleansing liquids, solutions, or gels. Additional devices corresponding to Foley catheter use, including towels 710, drapes 711, rubber gloves 712, one or more swab sticks 713, a Foley catheter securement device 714, a Foley catheter insertion tag 715, and a Foley insertion tag, which is a dated and/or time stamped label that is secured to the catheter tubing once the Foley catheter 701 is inserted, and so forth, can be disposed in the tray 100 as well. As an illustration of this flexibility, one or more towels can be disposed beneath the catheter assembly.

Syringes 706,707 are disposed in the first compartment 101. One syringe 706 contains a sterile liquid, such as sterile water. The other syringe 707 contains a lubricant, such as lubricating jelly. The lubricating jelly can be discharged into the first compartment 101 to lubricate at least a portion of the Foley catheter 701 when the Foley catheter 701 is passed from the second compartment 102 into the first compartment 101.

Once the necessary components are disposed within the tray 100, the tray 100 can be sealed with a wrap to keep the internal components sterile. The wrap can be any of a number of types of material. The wrap can be a central sterile reprocessing (CSR) wrap that is used widely by medical professionals in hospitals, ambulatory surgical centers, and the like during medical procedures. While a CSR wrap is one example of a wrap that can be used, other wraps, such as plastic, cotton, linen, paper, or combinations thereof, can be substituted for the CSR wrap.

The CSR wrap can be folded about the tray 100 for sealing and can be correspondingly unfolded to reveal the tray 100. Once unfolded, the CSR wrap can then be used in the catheter insertion process. For example, an unfolded CSR wrap can be used to provide a sterile field in which the tray 100 sits for unloading and subsequent use.

Printed instructions can then be attached to, disposed upon, or disposed within the tray 100. Instructional banners can be included as well. The instructional banners can be attached to the tray, attached to wrap layers positioned around the tray, or tucked within folds of the wrap layers positioned around the tray. The instructional information can inform medical personnel how to apply a Foley catheter to a patient. The instructional information positioned on the instructional banners can also inform medical personnel—or patients themselves—how to care for, clean, and properly maintain a Foley catheter when applied to a patient as well.

The printed instructions can include a health care services portion and a patient portion.

The health care services portion can include instructions telling the health care services provider, for example, how to set up a sterile or otherwise clean work environment, how to prepare the catheter assembly 700 disposed within the tray, how to use the other devices within the tray, how to insert the catheter, how to secure the drainage bag to the catheter, how to empty the drainage bag, how to obtain a urine sample, and so forth. The instructions can include pictures or illustrations showing visually how the various steps should be done as well.

The patient portion can include helpful suggestions or instructions for the patient. The patient portion can be detachably coupled to the health care services portion, such as by a perforated line that is easily torn to separate the patient portion from the health care services portion. Examples of suggestions or instructions that may be included in the patient portion include information on what a catheter is, what the patient should understand about the catheter, how to reduce the chance of getting an infection, information about infections commonly associated with catheters, symptoms of infections commonly associated with catheters, and suggestions for home use of the catheter assembly 700. The health care services portion may include an instruction for the health care services provider to detach the patient portion from the health care services portion and instructions to discuss the patient portion with the patient.

The health care services portion can tell the medical services provider how to perform a standard catheterization procedure. For instance, the tray 100 can be equipped with an adhesive label that can be used to identify the patient or specimen in the specimen container 708. Further, a label can be included to mark or otherwise identify the material in the fluid bag attached to the catheter. Such labels can include pre-printed fields, such as date, time and name Further the printed instructions can notify the medical services provider that the devices disposed within the tray 100 are ordered corresponding to use during the catheterization procedure.

The printed instructions can inform the medical services provider of special instructions.

For instance, the printed instructions can inform the medical services provider not to leave a catheter in a patient for more than forty-eight hours without a physician's approval. Where the printed instructions include such information, the labels included in the tray 100 may have pre-printed fields for the time of insertion that can be filled in by the medical services provider performing the catheterization procedure.

Once the printed instructions have been affixed to, or placed with, within, or atop the tray 100, the assembly can be sealed in a sterile wrap such as a thermally sealed bag. The thermally sealed bag can optionally include a preformed opening. For example, the opening can include one or more tabs that a health care services provider is instructed to pull to open the bag. Inclusion of a sterile wrap not only keeps the contents within the bag sterile, but also allows the instructions to be included with the tray assembly, yet outside the CSR wrap.

The printed instructions can be disposed atop the CSR wrap such that the health care services portion of the printed instructions is disposed on the top of the printed instructions, with the patient portion being disposed adjacent to the CSR wrap, such as when the printed instructions are configured as an accordion-style folded instruction pamphlet. While the printed instructions of can be configured as a folded, printed, separate article disposed atop the CSR wrap, rather than including separate printed instructions, the instructions for use can be printed on the CSR wrap as well.

Additional instruction materials may be included with the completed assembly as well.

For example, an adhesive instruction tag can be affixed to the sterile wrap. The instruction tag may be adhered to an outer packaging that encloses the tray, the sterile wrap material, or both. For example, the instruction tag can include information regarding whether a catheter procedure is needed. Text such as “Is there a valid clinical reason?” may be included under an instruction to “Stop” that includes the following information:
Before inserting the Foley catheter, at least one of the following conditions should exist:
Acute urinary retention or obstruction
Precise measurement of urinary output needed
Select surgical procedures
Open sacral or perineal wounds in incontinent patient
Prolonged immobilization
End of life care

Further, checklist text may be included, such as “Checklist for Foley Catheter Insertion” included under the word “Check” that includes the following information:
Check Each Box Upon Completion:
Obtain order from physician/provider
Document clinical reason for insertion
Explain procedure to patient
Use smallest catheter possible
Perform hand hygiene
Follow aseptic technique

Additional information may also be included, such as a fillable form that provides fields for the date and time of insertion of the catheter to be recorded, the name of the health care services provider, and the signature of the health care services provider. The above text for the instruction tag is illustrative only and may be customized as desired by the manufacturer.

Prior to depositing the packaged catheter assembly into outer packaging such as a surrounding bag, optional printed instructions can be attached to or disposed upon the packaged catheter assembly. As noted above, the printed instructions can include a health care services portion and a patient portion. The instructions can include pictures or illustrations showing visually how the various steps should be done as well.

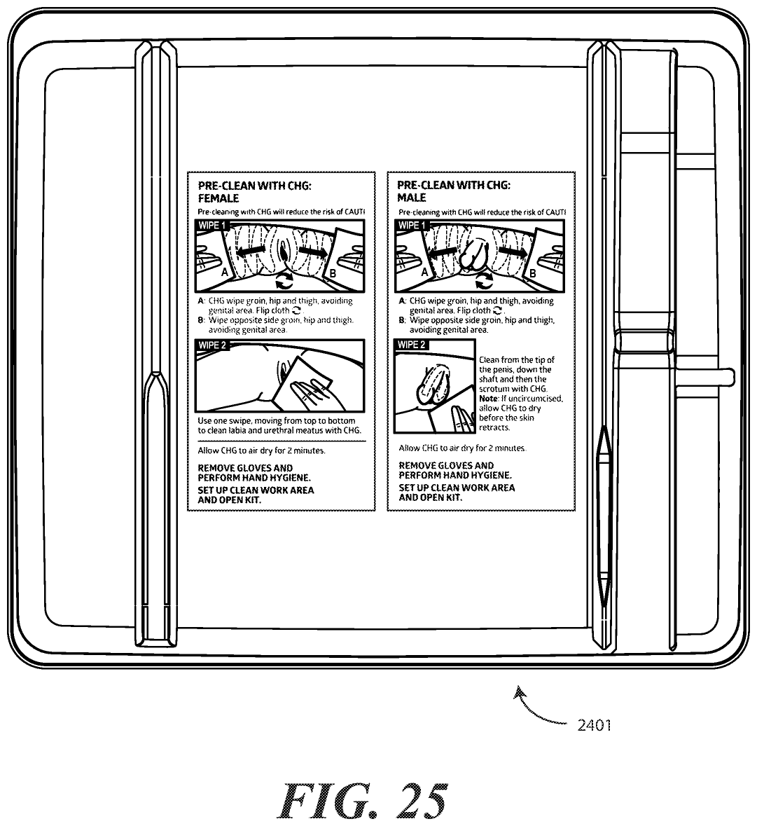

Once the printed instructions have been affixed to, placed with, or placed atop the packaged catheter assembly, the assembly can be sealed in a sterile wrap such as a bag, which may be thermally or otherwise sealed. The thermally sealed bag optionally includes a preformed opening. For example, in one embodiment, the preformed opening can include one or more tabs that a health care services provider is instructed to pull to open the bag. Inclusion of a sterile wrap not only keeps the contents within the bag sterile, but also allows the printed instructions to be included with the tray assembly, as will be described below with reference to FIGS. 25-28.

In one embodiment the printed instructions are disposed atop the one or more layers of wrap material such that the health care services portion of the printed instructions is disposed on the top of the printed instructions, with the patient portion being disposed adjacent to the one or more layers of wrap material. Additional instruction materials may be included with the completed assembly as well. For example, in one embodiment an adhesive instruction tag can be affixed to the bag.

The printed instructions can be configured as an instruction manual suitable for inclusion with a tray as described above. In one embodiment, the printed instructions are configured as a two-portion instruction manual having a health care services portion and a patient portion. This manual can be configured with a greeting card appearance. The patient portion can be detachably coupled to the health care services portion, and thus separated from the health care services portion by a perforation. For example, where the printed instructions are configured as a printed material on a paper-based stock, the perforation can be a perforated line running along a dimension of the printed instructions such that the printed instructions can be easily torn along the perforation to separate the patient portion from the health care services portion.

In one embodiment, the printed instructions are configured as a plurality of panels. In other embodiments, the printed instructions can be configured as an instruction manual that is formed with an accordion-style fold, with each of the panels forming a page of the instruction manual. Some panels can form the health care services portion, while other panels form the patient portion. Panels can be separated by the perforation with the patient portion being tearably separable from the health care services portion.

In one embodiment, the health care services portion includes instructions for using the catheter assembly and other corresponding medical devices disposed within the accompanying tray. The instructions can include text and/or figures or illustrations showing how to use the catheter assembly and corresponding medical devices on the patient, as well as instructions on preparation, taking samples, preventing infection, and so forth. The instructions, in one embodiment, also include an instruction to detach the patient portion, give the patient portion to the patient, as well as an instruction to discuss the information disposed on the patient portion with the patient.

Similarly, the patient portion may also include instructions and/or helpful suggestions for the patient who is undergoing the catheterization procedure. For instance, this information can include any one or more of the following: a description of what a catheter is, what the patient should know about the catheter, how to reduce the chance of getting an infection from the catheterization procedure, what infections commonly associated with catheterization procedures typically are, the symptoms associated with infections commonly associated with catheterization procedures, and information about using the catheter at home. Additionally, the patient portion may include custom information as well. For example, in one embodiment the patient portion includes an informational section configured such that the health care service provider's name and contact information can be written thereon. It will be clear to those of ordinary skill in the art having the benefit of this disclosure that the disclosure is not so limited. For example, additional types of health care service instructions or patient instructions or suggestions can also be included.

The panels, where included, can illustrate exemplary instructions for the health care services portion and patient portion of an illustrative instruction manual for a catheter assembly and corresponding medical devices included with an accompanying tray. It will be clear that these panels and the information printed thereon can be varied in any number of ways without departing from the spirit and scope of the disclosure as described herein and recited in the following claims. For example, the number of panels can be varied. Additionally, the information printed thereon can be condensed, expanded, or altered without departing from the spirit and scope of the disclosure. Also, the exemplary information may be moved from the panels shown to other panels, as a particular application may warrant.

In one embodiment, a panel will be configured such that when the catheter package assembly within which the instruction manual is disposed is initially opened, the panel will be readily viewable. In one or more embodiments, the panel will be viewable prior to removal of the CSR wrap.

The panel can include general information about the catheter assembly and corresponding medical devices disposed within the tray. For example, this information can include part number information, trade name information, and manufacturer information. A diagram of the contents of the package assembly may be included as well. Illustrating by example, the panel can illustrate a tray having a catheter assembly and corresponding medical devices disposed therein. The corresponding medical devices can include a pair of syringes and a specimen container. Additionally swab sticks, a catheter securement device, a Foley insert tag, vinyl gloves, a fenestrated drape, an underbuttocks drape, and hand sanitizer solution or wipe are disposed within the tray.

In addition to a diagram, the panel can also include a written description of the elements included in the tray. Further, sterility information can be included. The panel can even include instructional material on how to use the instruction manual as well.

Another panel can include instructions for using the catheter assembly and the corresponding medical devices. The panel can include instructions for setting up a clean work area. The instructions can include text, pictures, illustrations, or combinations of these.

In one embodiment, the instructions for setting up a clean work area include a hygiene performance step, which may include instructions to wash hands, optionally put on gloves (which at this step can be non-sterile gloves), and so forth. The instructions may then include information on opening the remainder of the catheter package assembly. For instance, the instructions can indicate that the health care provider should remove the CSR wrap folded about the tray. In one or more embodiments, removal of the CSR wrap by unfolding creates a sterile field about the tray.

This panel can then instruct the health care provide to pick up the underbuttocks of the patient and to place the underbuttocks wrap beneath the patient. The panel can then instruct the health care provider to use the hand sanitizing solution provided with the catheter package assembly.

The various panels may include suggestions for preventing a catheter associated urinary tract infection. In addition to information for setting up a clean work area, in one embodiment a panel includes instructions for preparing the catheter assembly as well. For example, a panel can instruct the health care services provider to don sterile gloves, as the hands were sanitized previously. The panel can then tell the health care services provider to place the fenestrated drape with a shiny side down on the patient without contaminating the sterile gloves. The panel can then instruct the health care services provider to test the balloon of the catheter assembly with the water-filled syringe stored in the first compartment. The panel can then instruct the health care services provider to leave the syringe connected to the catheter assembly.

The panel can then provide instructions on using the first compartment of the tray as a lubricant application chamber as described above. Specifically, in this illustrative embodiment, the panel instructs the health care services provider to inject the lubricating jelly found in the second syringe of the first compartment into the first compartment. The panel can also instruct the health care services provider to pass the tip of the catheter through the first opening in the wall separating the first compartment and second compartment into the lubricating jelly, thereby lubricating the tip of the catheter.

Another panel can include instructions printed thereon continue to provide the health care services provider with information regarding use of the catheter assembly. For example, in one embodiment, this information includes instructions on inserting the catheter.

The panel can direct the health care services provider to tear open the swab stick package and to use the swab sticks to clean the patient from the top down. The instruction can note that each swab stick is intended for one use only to properly maintain the sterile field. The panel can direct the health care services provider to initiate the catheterization process by inserting the catheter assembly into the patient.

The panel can direct the health care services provider to secure the drainage bag to the catheter assembly. The panel can direct the health care services provider to clean up upon completion of the catheterization process. The panel can provide instructions on completing the label on the Foley insertion tag included with the catheter package assembly and attaching it to the tubing or drain bag attached to the catheter assembly.

The health care services provider can be instructed to detach the patient portion from the health care services portion by tearing the two apart along the perforation. The panel can further instruct the health care services provider to discuss the patient information printed upon the patient portion with the patient. The panel can instruct that documentation of the entire procedure should be completed.

Another panel can provide a first side of the patient portion. This panel can include information describing what a catheter is and why a catheter might be used. The panel also can include information describing what the patient should know regarding catheters and catheter use. For example, this information might notify the patient that the health care services provider should wash hands prior to inserting the catheter, and that it is acceptable to ask them to do so if they have not done so before the patient.

The panel can also include information regarding how the patient can reduce the chances of getting an infection. This information can include a statement that the patient should wash their hands prior to touching the catheter assembly. The information may also include a statement that the drainage bag should always be kept at a level beneath the patient's navel, and that the patient should inform a helper when the bag is more than half full.

Another panel can form the second side of the patient portion of the instruction manual, and accordingly, can include additional information that a patient may wish to know when using a catheter assembly. By way of example, information informing the patient as to what common infections associated with catheter use are and how they are contracted can be included. Information can provide symptoms of these common infections, such as fever, blood in the urine, burning or painful urination, or frequent or more urgent urination after catheter removal. Information can inform the patient of what they should know prior to going home after a catheter procedure.

The information can comprise an informational section configured such that a health care provider's name and contact information may be written thereon. This is helpful to the patient in the event that the symptoms recited in information should arise after the procedure, in that the patient has readily available access to the information required to contact a physician or other health care provider. An advantage of having this information on the patient portion when the patient portion is detachable is that the patient can take it with them upon completion of the procedure.

Other panels can provide additional health services information. For example, information for emptying the drain bag and information describing how to obtain a urine sample can be included. Other information suitable for inclusion with the instructional materials, instructional inserts, and/or instructional banners will be described below. Still other information will be obvious to those of ordinary skill in the art having the benefit of this disclosure.

A method for manufacturing a packaged catheter assembly in accordance with embodiments of the embodiments of the disclosure can include providing a tray (100) having at least a first compartment (101) for accommodating one or more syringes (706,707) and a second compartment (102) for accommodating a flexible medical device, such as a Foley catheter assembly (700). As noted above, in one embodiment the first compartment (101) will have a first compartment base member (107) having an inclined, stair-stepped contour (115). The first compartment (101) and second compartment (102) can be separated by a first barrier (105) having an opening (121) therein.

Once the tray (100) is procured, the manufacturer can dispose at least one syringe (706) in the first compartment (101). Optionally, the manufacturer may include additional components with the tray (100). For example, a catheter securement device, a Foley insert tag, or other complementary components may be included.

In one embodiment, a second syringe (707) will be disposed in the first compartment (101). In another embodiment, the second syringe (707) will be disposed laterally within the first opening (121) and, where present, a second opening (122).

The manufacturer can then place the catheter assembly (700) in the second compartment (102). Other components may be disposed in the tray (100) as well, including a specimen container (708) that is disposed in a third compartment (103). Further, other devices may be included, such as towels, drapes, printed instructions, one or more antiseptic packets, and so forth. These other devices can be disposed in various compartments within the tray (100).

The tray (100) can then be sealed. This can be accomplished by folding a CSR wrap about the tray (100). In such an embodiment, the CSR wrap can be used during the catheter insertion procedure as well. Optionally, the manufacturer can enclose printed instructions, instructional banners, or other instructional information. In one embodiment, the printed instructions will direct a user to discharge contents of at least one syringe into the first compartment (101) and to pass at least a portion of the catheter assembly (700) through the opening and into the contents to lubricate the catheter.

The manufacturer can place a sterile wrap about the tray (100) and the printed instructions, where included. A sticker or other sealing device can be applied that indicates the contents to be sterile as well. The completed assembly can be shipped to a medical services provider.

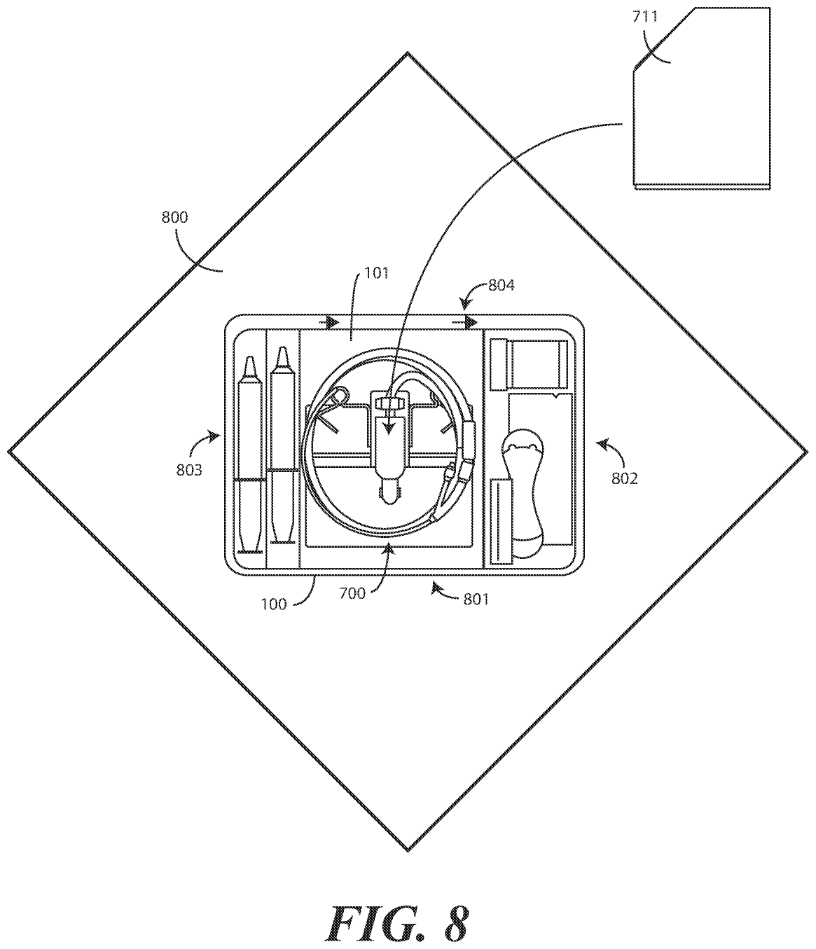

Turning now to FIGS. 8-15, illustrated therein is a method of packaging a Foley catheter assembly and corresponding tray in accordance with embodiments of the disclosure. FIGS. 8-15 illustrate one exemplary method graphically, which each figure representing one or more steps of the method, as the illustrations serve to better explain these steps than would a flow chart or other diagram. While FIGS. 8-15 illustrate one method of packaging a tray and Foley catheter assembly, it will be clear to those of ordinary skill in the art having the benefit of this disclosure that other methods can be used as well.

Further, in creating this article of manufacture, i.e., the packaged Foley catheter assembly, the steps of FIGS. 8-15 may be either manual or automated. A person can execute the steps to create the article of manufacture in one embodiment. Alternatively, industrial machinery, equipment, and robotics can be designed and programmed to execute the steps with the assistance of one or more processors and executable instructions stored in memory.

Beginning with FIG. 8, in this step, a tray 100 is provided. The tray includes at least one compartment, such as the first compartment 101 that is configured for receiving the Foley catheter assembly 700. As described above, the tray 100 can include additional compartments as well, such as those for receiving syringes, specimen jars, and so forth.

At this step, the Foley catheter assembly 700 is placed within the first compartment 101 as previously described. A folded drape 711 can optionally be placed atop the tray 100.

The tray is then placed upon one or more layers of wrap material 800. In one embodiment, the wrap material 800 can be CSR wrap. For example, in the illustrative embodiment of FIG. 8, the wrap material 800 comprises a white layer of CSR wrap measuring 24 inches square. As previously noted, other materials can be used as well, including plastic materials, cotton materials, paper materials, synthetic materials and so forth. The wrap material 800 can be of different shapes and sizes as well.

While the tray 100 can be sealed with a simple layer of plastic adhered to the top of the tray 100, providing the wrap material 800—in addition to the plastic adhered to the top of the tray 100 or instead of the plastic adhered to the top of the tray 100—can be advantageous in many applications. For example, when the wrap material 800 is a medically usable material, such as CSR wrap, a medical services provider may unfold the wrap about the tray 100 to create a sterile field for the Foley catheterization procedure. For this reason, one or more layers of wrap material 800 are simply folded about the tray 100 in this illustrative embodiment.

Note that for reference and ease of explanation, the tray 100 will be described as having four sides: a first side 801, a second side 802, a third side 803, and a fourth side 804. As these sides will not be visible in every view, due to the folding of the wrap material 800 about the tray 100, they are initially noted here. Note that four sides are used because the illustrative tray 100 is rectangular in shape. Were the tray a triangle, there would be three sides. Were the tray ovular or circular, there would be an infinite number of sides.

Turning now to FIG. 8, at this step a first portion 901 of the one or more layers of wrap material 800 is folded about a first side (801) of the tray 100. In this illustrative embodiment, the tray 100 is oriented at a rotation of approximately forty-five degrees relative to the one or more layers of wrap material 800, with both the wrap material 800 and the tray 100 being rectangular in shape. As such, the first portion 901 comprises a first corner of the wrap material 800. It will be clear to those of ordinary skill in the art having the benefit of this disclosure, however, that embodiments of the disclosure are not so limited. For example, the wrap material 800 can be configured as a circle or oval. Executing the step shown in FIG. 23, a first portion of that material could be folded about a first side of the tray 100 in similar fashion.

As shown in this figure, an optional step can also be included in the method of packaging the Foley catheter assembly. To wit, in one or more embodiments, once the first portion 901 of wrap material is folded about the first side (801) of the tray, a package of skin sanitizer 709 and a package of rubber gloves 712 can be placed atop the first portion 901 of the wrap material 800. In one or more embodiments, this placement occurs after the first portion 901 of the wrap material 800 has been folded about the first side (801) of the tray 100.

As noted above, in one embodiment the one or more layers of wrap material 800 can be unfolded to create a sterile field about the tray 100. A patient can be placed atop this sterile field for the catheterization procedure. Even if the surface below the wrap material 800 is also sterile, the use of the wrap material 800 as a foundation for the procedure further ensures that the sterile field will not be breached.

To help ensure that the health care provider does not inadvertently breach the sterile field, in one embodiment a package of liquid skin sanitizer 709 or other cleanser and/or a package of rubber gloves 712 may be included. In such an embodiment, upon opening the packaged catheter assembly, the health care services provider may—before ever touching the catheter assembly or tray contents—apply the liquid skin sanitizer 709 to their hands and don rubber gloves 712. The inclusion of these accessories in the packaging eliminates the need for the health care services provider to have to leave the sterile field to wash their hands, obtain gloves, and so forth.

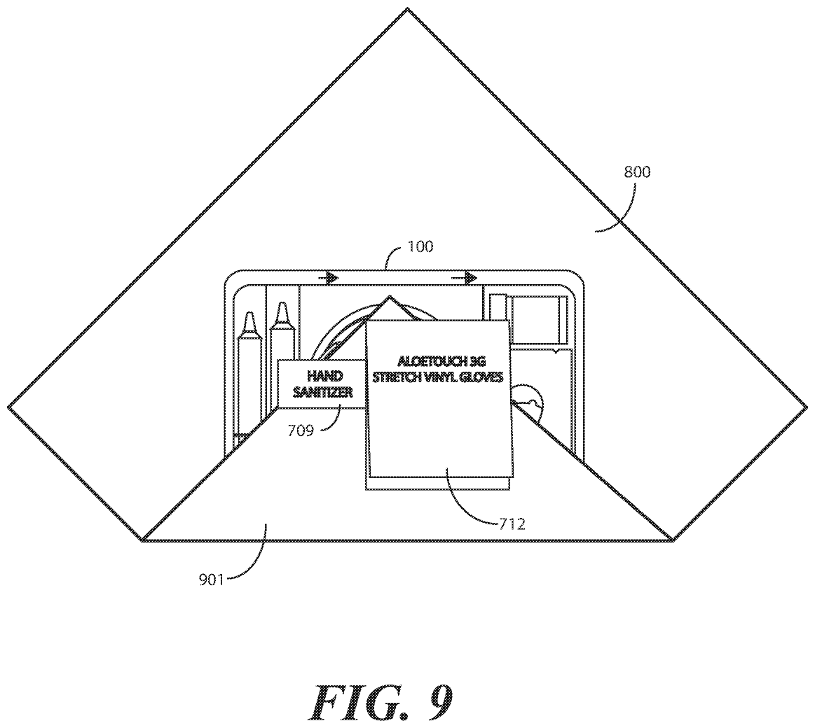

In the illustrative embodiment of FIG. 9, the package of liquid skin sanitizer 709 and package of rubber gloves 712 are simply placed atop the first portion 901 of the one or more layers of wrap material 800. In one or more embodiments, these implements are held in place by other portions of the one or more layers of wrap material 800 by way of subsequent folding steps. Other methods of holding them in place, including light adhesives or the design of pockets in the one or more layers of wrap material 800 may also be used.

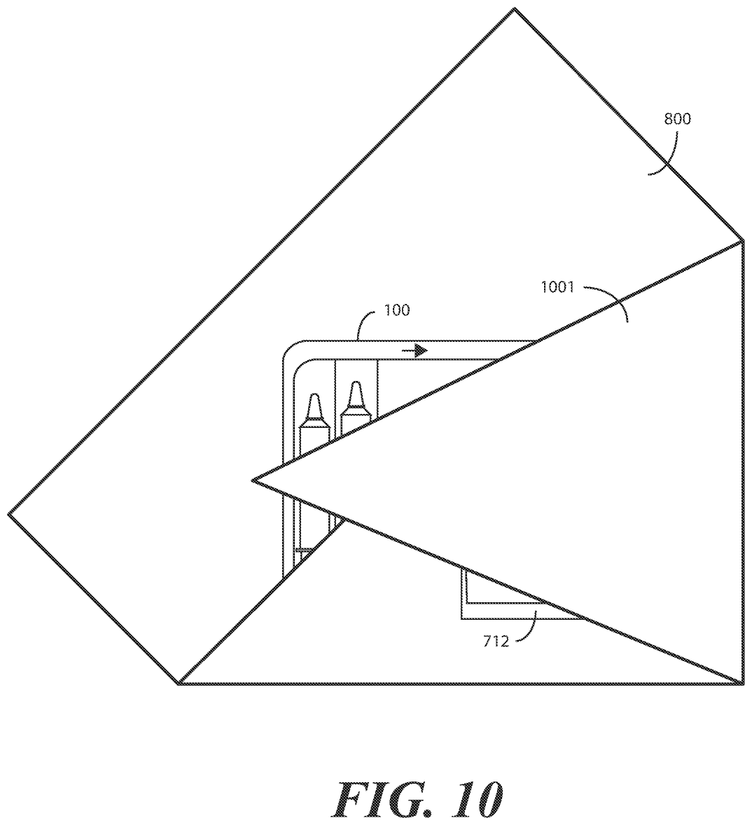

Turning now to FIG. 10, at this step a second portion 1001 of the one or more layers of wrap material 800 is folded about a second side (802) of the tray 100. Where the optional package of liquid skin sanitizer (709) and package of rubber gloves 712 are included, the second portion 1001 of the one or more layers of wrap material 800 may be folded so as to cover or partially cover these items.

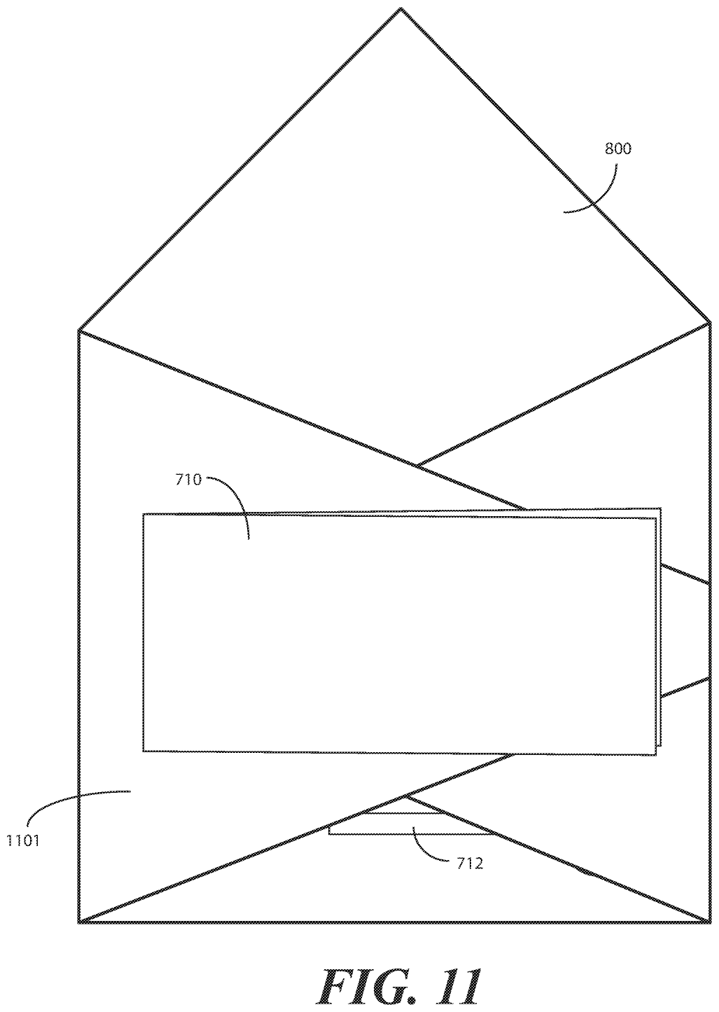

Turning now to FIG. 11, at this step of the method a third portion 1101 of the one or more layers of wrap material 800 is folded about a third side (803) of the tray (100). Where the optional package of liquid skin sanitizer (709) and package of rubber gloves 712 are included, the third portion 1101 of the one or more layers of wrap material 800 may be folded so as to cover or partially cover these items. One or more towels 710 can optionally be placed atop the third portion 1101 of the one or more layers of wrap material 800 as well.

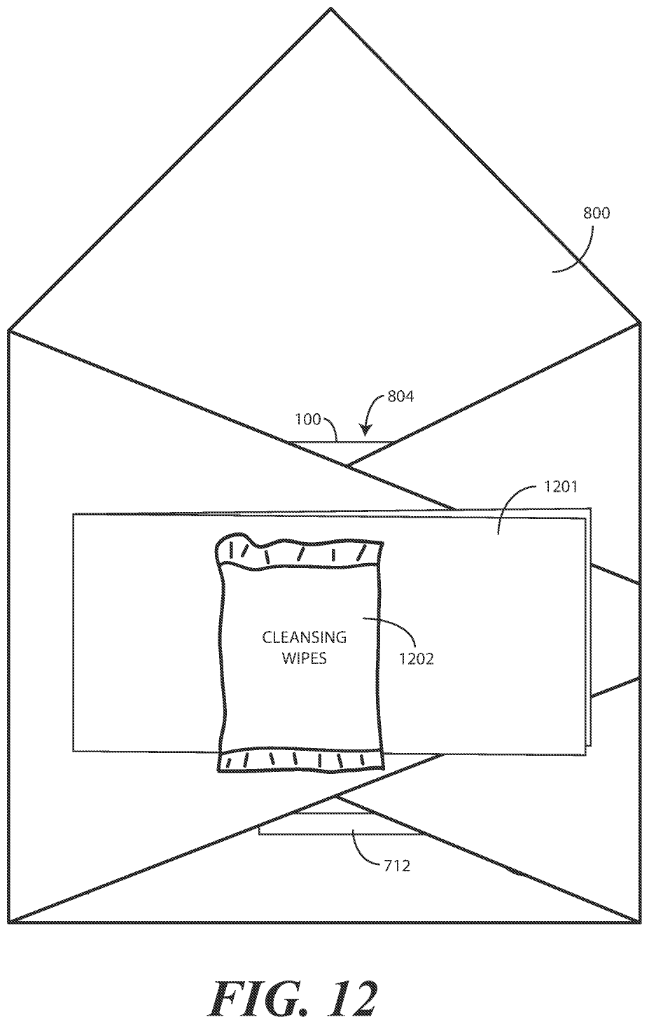

Turning now to FIG. 12, illustrated therein is another optional step of the method of packaging the Foley catheter assembly and tray. In many Foley catheterization procedures, a first layer of material will be placed under the patient, while a second layer of material is placed atop the patient. For such applications, the packaged catheter assembly can include an additional layer of wrap material 1201. In the illustrative embodiment of FIG. 12, the additional layer of wrap material 1201 comprises a folded layer of CSR wrap measuring 17 by 17.5 inches. The additional layer of wrap material 1201 in this illustrative embodiment is folded as a 4 by 2 matrix.

The one or more layers of wrap material 800 and the additional layer of wrap material 1201 can be the same type of material. Alternatively, the one or more layers of wrap material 800 and the additional layer of wrap material 1201 can be different. In one embodiment, for example, the additional layer of wrap material 1201 can be a fenestrated wrap with one or more pre-formed openings suited to the catheterization procedure.

In one embodiment, the additional layer of wrap material 1201 is configured to be visibly distinguishable from the one or more layers of wrap material 800. For example, in one embodiment, the additional layer of wrap material 1201 is a different color than the one or more layers of wrap material 800. The one or more layers of wrap material 800 can be white, for instance, while the additional layer of wrap material 1201 can be light blue or light green. Other color combinations can equally be used.

As with the package of liquid skin sanitizer (709) and package of rubber gloves 712, in one embodiment the additional layer of wrap material 1201 can be placed atop portions of the one or more layers of wrap material 800. In such an embodiment, the additional layer of wrap material 1201 can be held in place by way of subsequent folding steps, as the additional layer of wrap material 1201 is disposed along other folded portions of the one or more layers of wrap material 800 prior to folding a fourth portion of the one or more layers of wrap material about the fourth side 804 of the tray 100. One or more cleaning wipes 1202 can optionally be placed atop the additional layer of wrap material 1201 as well.

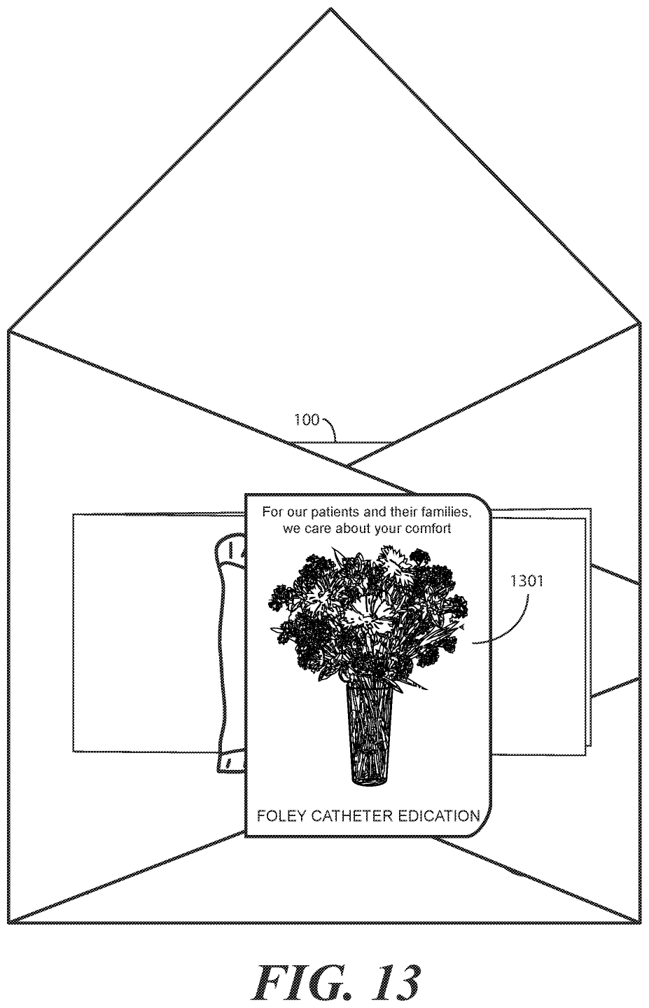

Turning now to FIG. 13, illustrated therein is another optional step that can be included in the assembly of a Foley catheter medical kit configured in accordance with one or more embodiments of the disclosure. Specifically, a patient aid 1301 can optionally be placed atop the portions of the one or more layers of wrap material 800 that have thus far been folded about the tray 100.

As shown in FIG. 13, in one or more embodiments the patient aid 1301 is configured as a standalone device suitable for delivery to patients. In such an embodiment, the patient aid 1301 is physically separate from health care provider information, which is included in or at other locations in a medical procedure kit. In one embodiment, the patient aid 1301 is designed for inclusion with a medical procedure kit, such as the one shown in FIG. 7 above.

In one embodiment, the patient aid 1301 is configured as an educational card or pamphlet comprising a first portion and a second portion, both of which are either carried on, disposed, on or coupled to a carrier. The first portion is an outwardly visible portion, while the second portion faces inwardly. In one embodiment, the first portion is configured with a disguise so as to be any of aesthetically pleasing, entertaining, and/or comforting in appearance. In one embodiment, the second portion is configured as an education portion.

In one embodiment, the patient aid 1301 is associated with medical procedures and/or medical devices. Accordingly, the second portion can be configured to have an informational set disposed thereon related to educating the intended recipient. The informational set can comprise any of the following: educational information corresponding to a medical procedure, patient care information corresponding to a medical procedure, information relating to a medical device, such as a urinary catheter, peripherally inserted central catheter, or wound dressing, that is applied to the patient, an illustrated guide depicting patient care for medical devices, or combinations thereof.

In the illustrative embodiment of FIG. 13, the patient aid is configured as a folded card that has the first portion facing outwardly and the second portion facing inwardly. It will be clear to those of ordinary skill in the art having the benefit of this disclosure that other folding configurations could also be used, including tri-folds, quad-folds, and so forth. In one embodiment, the first portion and the second portion are detachable from each other. In another embodiment, they are integrated with the carrier so as to not be readily detachable.

To provide an aesthetically pleasing, entertaining, and/or comforting appearance, in one embodiment the outward facing portion is configured with a greeting card appearance, while the inward facing portion comprises patient information relating to a medical procedure, medical device, post-procedure medical care, or post-procedure medical device care. In another embodiment, configured primarily for children, the outward facing portion is configured as an activity card, with the inward facing portion being configured with the patient information, which includes educational information corresponding to a medical procedure. The patient aid 1301 provides the patient, or those associated with assisting the patient, with education on the procedure performed on the patient as wells as care instructions for the patient or relative to administer subsequent to leaving professional care.

To further provide an aesthetically pleasing, entertaining, and/or comforting appearance, the first portion of the patient aid 1301 can be configured in a variety of ways. For example, in one embodiment, the first portion includes one or more healing colors disposed thereon or integrated therein. In another embodiment, the first portion has a greeting disposed thereon. In one embodiment, the greeting is configured in large-font type, i.e., fonts in excess of 14-point fonts, so as to be readily readable by a person with less than perfect eyesight.

In one embodiment, the first portion comprises a picture, which can be a serene landscape, flowers, candy, animals, and so forth. These features work to make the patient aid 1301 different in appearance so that it stands out to health care services providers. The features also work to create an emotional connection with the health care services provider, as well as appositive reaction from the patient. Additionally, even if a patient or health care services provider places the patient aid 1301 on a bedside table, the outward appearance increases the chance that family members will also read the patient information.

In one exemplary embodiment, the patient aid 1301 has the esthetics of a greeting card, such as a “get well soon card” for example. It is understood that a greeting card is one way of presenting the first portion of the patient aid 1301 to induce a caregiver to present the “greeting card” to the patient. It is the outward appearance of the patient aid 1301 in one embodiment, which is other than something that is generally related to the contents of the package it is carried in, which induces the caregiver to deliver the patient aid 1301 to the proper recipient. Said differently, the greeting card appearance is configured to provide a caregiver a visual indicator that the patient aid 1301 is intended for a patient. A greeting card look and feel thus forms one illustrative embodiment.

As noted above, the patient aid 1301 does not necessarily have a greeting card look. For example, in another embodiment the patient aid 1301 can be configured as a pediatric patient aid. In such a configuration, the picture on patient aid 1301 may include a caricature or cartoon character. This may be more appealing to children, and even some adults, than is the greeting card appearance. Medical procedure kits that may be used with children may include a patient aid that has cartoon characters and in one embodiment the character coincides with one or more current popular cartoon characters.

In the illustrative embodiment of FIG. 13, the patient aid 1301 includes at least one healing color and a greeting, which is configured in this embodiment as an inspirational phrase. Next, the outwardly facing portion includes an aesthetically pleasing image. The aesthetically pleasing image of FIG. 13 is a depiction of a vase of flowers, although it will be clear to those of ordinary skill in the art having the benefit of this disclosure that embodiments of the invention are not so limited. Other aesthetically pleasing images include puppies, sunsets, mountain streams, and so forth. The bottom of the outwardly facing portion in this illustrative embodiment includes in identifier that tells the patient the purpose of the patient aid 1301.

As noted above, the inwardly facing portions can include patient information. For example, in one embodiment where the patient aid 1301 is to be included with a catheter assembly, the interior includes the following illustrative text:

Here is some simple information about foley catheterization:

1. What is a Urinary Catheter?

A thin flexible tube that drains urine from the bladder into a collection bag. The catheter helps:

When you can't urinate.

To measure how much urine you're producing.

During and after some surgeries or tests.

2. What should you know about your catheter?

Only a trained technician inserts a catheter when necessary, and it is removed as soon as possible.

Caregivers must wash hands with soap or use alcohol-based rubs before and after touching your catheter.

If your caregivers don't clean their hands, politely ask them to.

Do not disconnect the catheter yourself

Inquire every day whether you still need the catheter.

3. What is ‘catheter-associated’ urinary tract infection (CAUTI)?

If a catheter introduces ‘outside’ germs into your urinary tract, they can cause an invention. If a UTI is acquired, you may experience:

Sudden fever and/or bloody urine.

Burning or painful urination, or pain below the stomach.

Frequent, or more urgent, urinating after catheter is removed.

Tell your provider right away. An antibiotic may be needed.

4. Can you reduce your chances of an infection? Absolutely!

Wash your hands before and after touching your catheter.

Make sure the tube is secured to your leg. Never twist, or tug on it.

Always keep the collection bag below the level of your belly button.

Do not disconnect the catheter yourself

Ask your doctor every day whether you still need the catheter.

In accordance with the examples above, the patient information can include educational information corresponding to a medical procedure, patient care information corresponding to a medical procedure, information relating to a medical device applied to a patient, an illustrated guide depicting patient care for the medical device, instructions for patient-administered care, combinations thereof, or other medical educational information.

The illustrative information set forth above can be printed in multiple languages, such as in Spanish or in English. Where two languages are used, the back portion may be the same as the image shown in FIG. 13, but with the greeting and optional identifier being set forth in a different language. Further, alternatives and variations of the information can be substituted for the example set forth above.

In alternative types of patient aids, a patient aid can be die cut in the shape of a door. The door can include windows, a knob, and a wood grain paneling aesthetic. An identifier configured to provide a caregiver a visual indicator that the patient educational card is intended for a patient is disposed at the top of the door, while a greeting is disposed at the bottom of the door. When the patient opens the door, the patient educational information is found therein.

In another embodiment, a patient aid can be configured as a pediatric patient aid. In this embodiment, the outwardly facing portion can be configured as an activity card. The activity card can include a cartoon, one example of which is a caricature of a young lion named “Buddy the Brave.”

In one embodiment, the cartoon defines an activity suitable for completion by a recipient.

Illustrating by example, Buddy can be shown juggling three balls. However, one of the balls can be shown as a blank. The activity card of this illustrative embodiment includes a sticker suitable for attachment to the activity card. Accordingly, to give Buddy three balls to juggle, the activity defined by the cartoon comprises attachment of the sticker to the activity card.

To encourage patients to be brave, an inspirational phrase can be disposed on one or both of the sticker and/or the activity card. In one or more embodiments, the sticker includes the inspirational phrase that says, “I′m Brave Like Buddy,” and this forms an indication that a recipient of the sticker is brave like a character depicted on the activity card. Either of the sticker or activity card could correspondingly include a request to “be brave,” such as “Be Brave Like Buddy,” or “Can You Be as Brave as Buddy?” In one or more embodiments, the sticker, like the activity card, also includes a depiction of Buddy.

While a fanciful animal, Buddy, can be included, it will be clear to those of ordinary skill in the art having the benefit of this disclosure that embodiments of the disclosure are not so limited. The cartoon or caricature could take other fanciful forms, including cartoon characters, superheroes, other animals, fanciful characters, and so forth. Additionally, the names, inspirational phrases, and other features could be different.

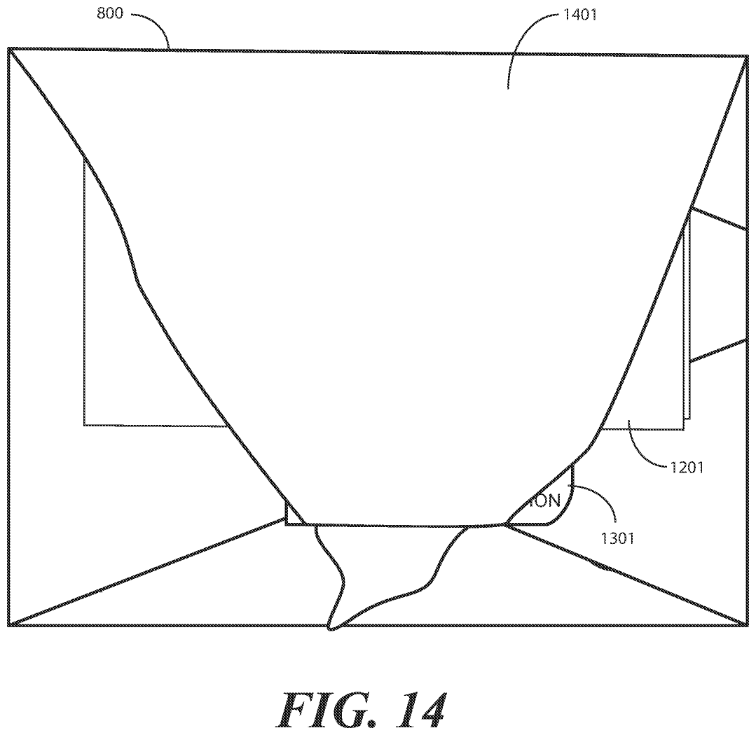

Turning now to FIG. 14, the tray (100) is enclosed in the one or more layers of wrap material 800 by folding a fourth portion 1401 of the one or more layers of wrap material 800 about a fourth side (804) of the tray (100) and then tucking at least one of the first portion (901), the second portion (1001), the third portion (1101), or the fourth portion 1401 of the one or more layers of wrap material 800 beneath at least another of the first portion (901), the second portion (1001), the third portion (1101), or the fourth portion 1401 of the layer of wrap material 800. In the illustrative embodiment of FIG. 14, a part of the fourth portion 1401 is tucked beneath parts of each of the first portion (901), the second portion (1001), and the third portion (1101). This step of tucking encloses both the additional layer of wrap material 1201 and the package of liquid skin sanitizer (709), the package of gloves (712), and the patient aid 1301 (where included) within the one or more layers of wrap material 800.

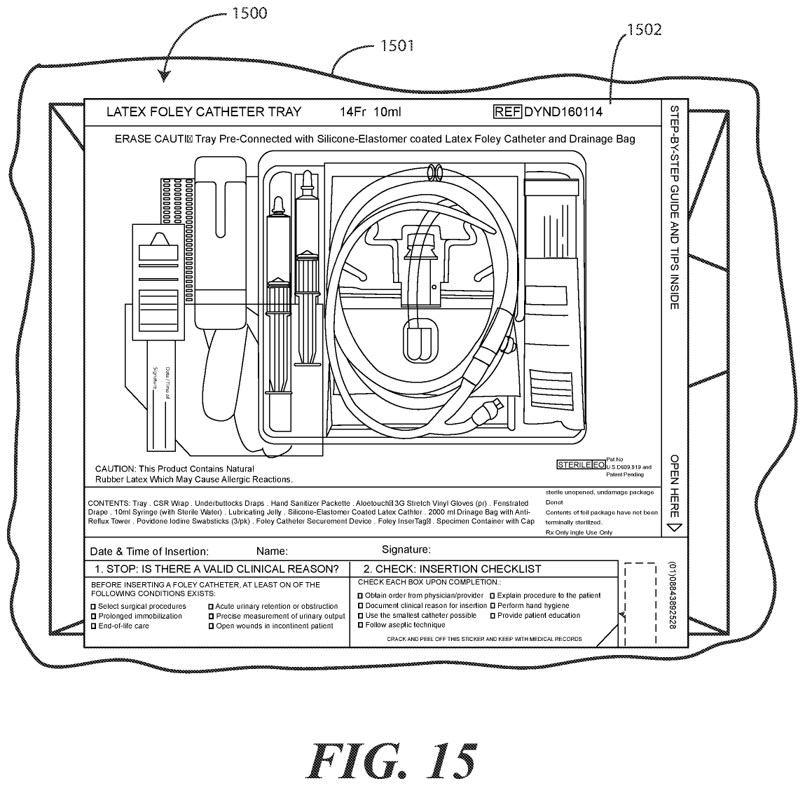

Turning now to FIG. 15, the wrapped Foley catheter assembly 1500 from FIG. 15 can be sealed in a bag 1501. Prior to depositing the packaged Foley catheter assembly 1500 into the bag 1501, optional printed instructions 1502 can be attached to or disposed upon the packaged Foley catheter assembly 1500 as well. The printed instructions 1502 can include a health care services portion and a patient portion as described above. The instructions can include pictures or illustrations showing visually how the various steps should be done as well.

Once the printed instructions 1502 have been affixed to, or placed with or atop the packaged Foley catheter assembly 1500, the assembly can be sealed in a sterile wrap such as a bag 1501, which may be thermally or otherwise sealed. The thermally sealed bag 1501 optionally includes a preformed opening. For example, in one embodiment, the preformed opening can include one or more tabs that a health care services provider is instructed to pull to open the bag 1501. Inclusion of a sterile wrap not only keeps the contents within the bag sterile, but also allows the printed instructions 1502 to be included with the tray assembly, yet outside the one or more layers of wrap material (800).

As noted above, trays configured in accordance with one or more embodiments of the disclosure can include one or more instructional banners. The instructional banners can be attached to the tray, attached to wrap layers positioned around the tray, or tucked within folds of the wrap layers positioned around the tray. The instructional information can inform medical personnel how to apply a Foley catheter to a patient. The instructional information positioned on the instructional banners can also inform medical personnel—or patients themselves—how to care for, clean, and properly maintain a Foley catheter when applied to a patient as well.

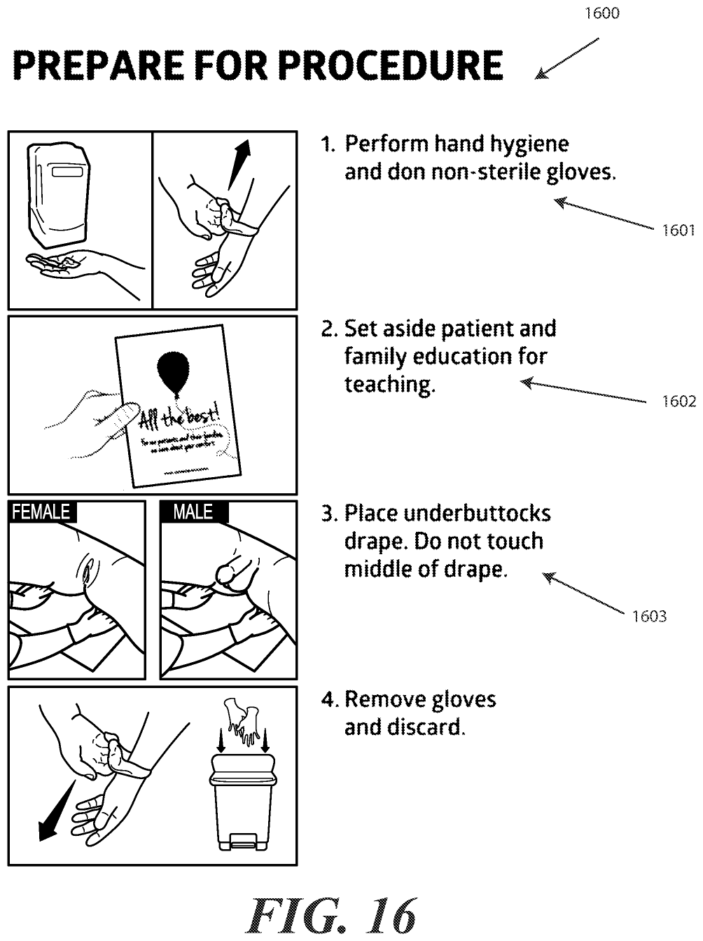

Turning now to FIG. 16, illustrated therein is one such instructional banner 1600. In this illustrative embodiment, the instructional banner 1600 includes instructions for using a Foley catheter assembly and the corresponding medical devices. As can be seen from this illustrative embodiment, the instructional banner 1600 can include instructions for setting up a clean work area. The instructions can include text, pictures, illustrations, or combinations of these.

In one embodiment, the instructions for setting up a clean work area include a hygiene performance step 1601, which may include instructions to wash hands, optionally put on gloves (which at this step can be non-sterile gloves), and so forth. The instructions may then include information regarding using the remainder of the Foley catheter package assembly. For instance, an instruction distribution step 1602 can include an instruction for a health care services provider tp deliver a patient aid to a patient. This step 1602 can further instruct the health care services provider to discuss the patient information printed upon the patient aid with the patient.

A wrap instruction step 1603 can instruct the health care provider to pick up the underbuttocks of the patient and to place the underbuttocks wrap beneath the patient. A glove disposal step can instruct the health care provider to remove the rubber gloves and discard the same.

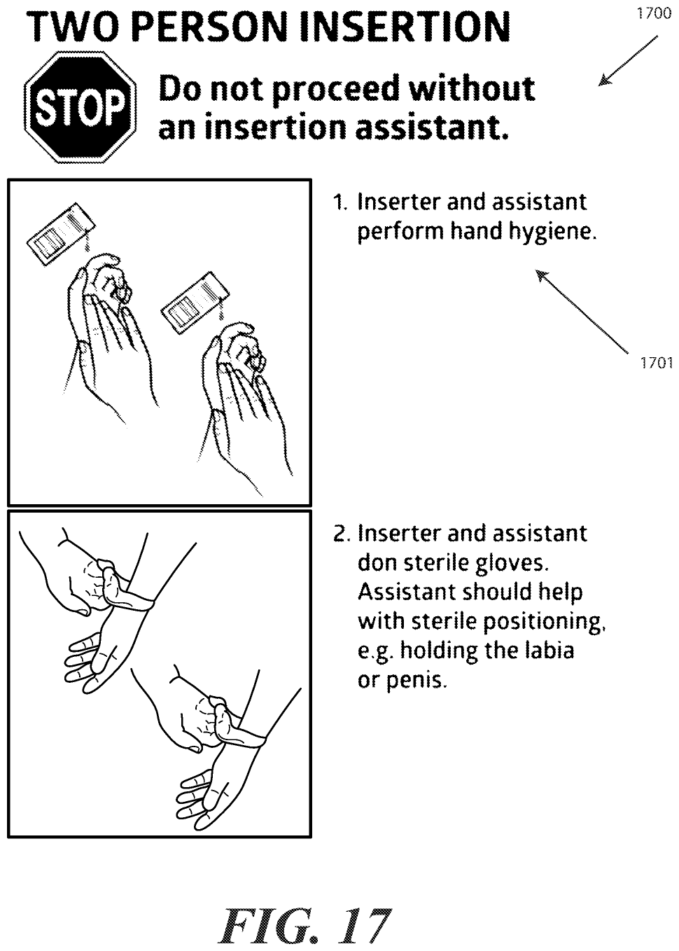

Turning now to FIG. 17, illustrated therein is another instructional banner 1700 configured in accordance with one or more embodiments of the disclosure. This instructional banner 1700 instructs two health care providers how to insert a Foley catheter. A hand preparation step 1701 can instruct the health care provider(s) to perform hand hygiene by applying the liquid skin sanitizer provided with the Foley catheter medical kit. A glove donning step 1701 instructs the health care services provider to don sterile gloves, as the hands were sanitized at step 1701.

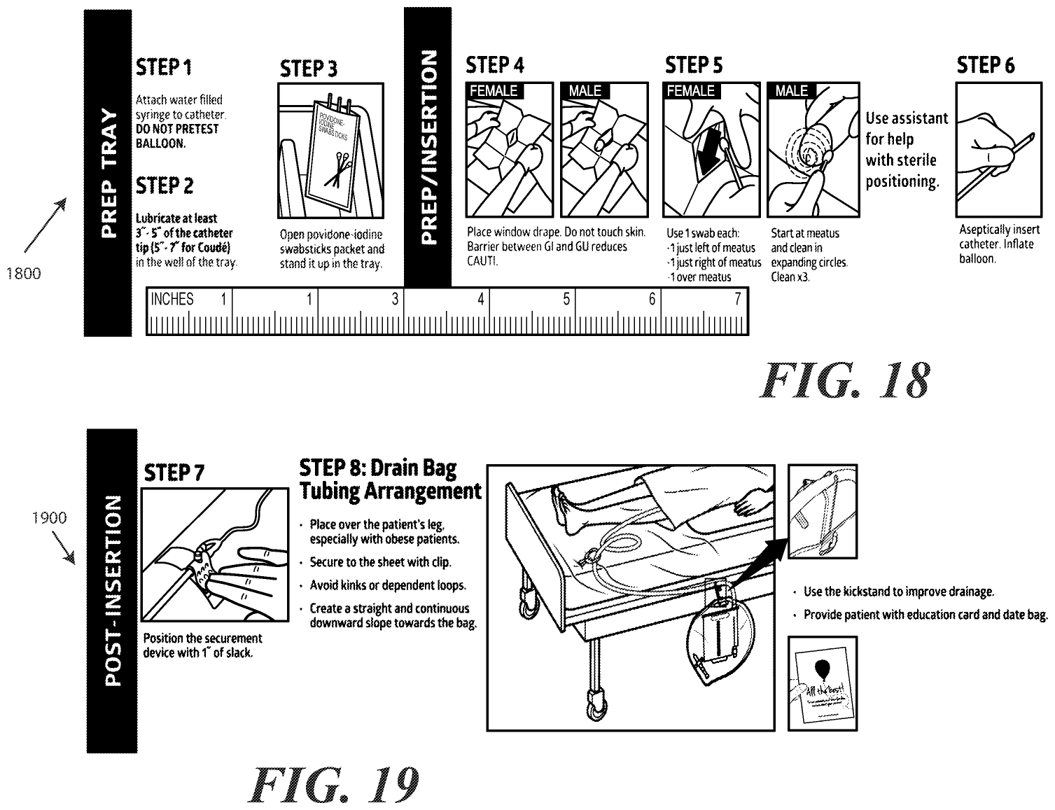

Turning now to FIG. 18, illustrated therein is another instructional banner 1800 configured in accordance with one or more embodiments of the disclosure. In the illustrative embodiment of FIG. 18, the instructional banner 1800 instructs health care personnel how to prepare a tray (100) such as the one described above with reference to FIG. 7. The steps included in this instructional banner 1800 can include attaching a water filled syringe (707) to the Foley catheter, lubricating a portion of the Foley catheter, cleaning the perineal portions of the patient, and so forth.

Turning now to FIG. 19, illustrated therein is another instructional banner 1900 configured in accordance with one or more embodiments of the disclosure. The illustrative instructional banner of FIG. 19 is a post-insertion instructional banner. The instructional banner 1900 informs a health care services provider how to position a Foley catheter securement device on a patient, how to arrange the tubing coupling the Foley catheter to the fluid drain bag, and how to position the fluid drain bag on a patient bed.

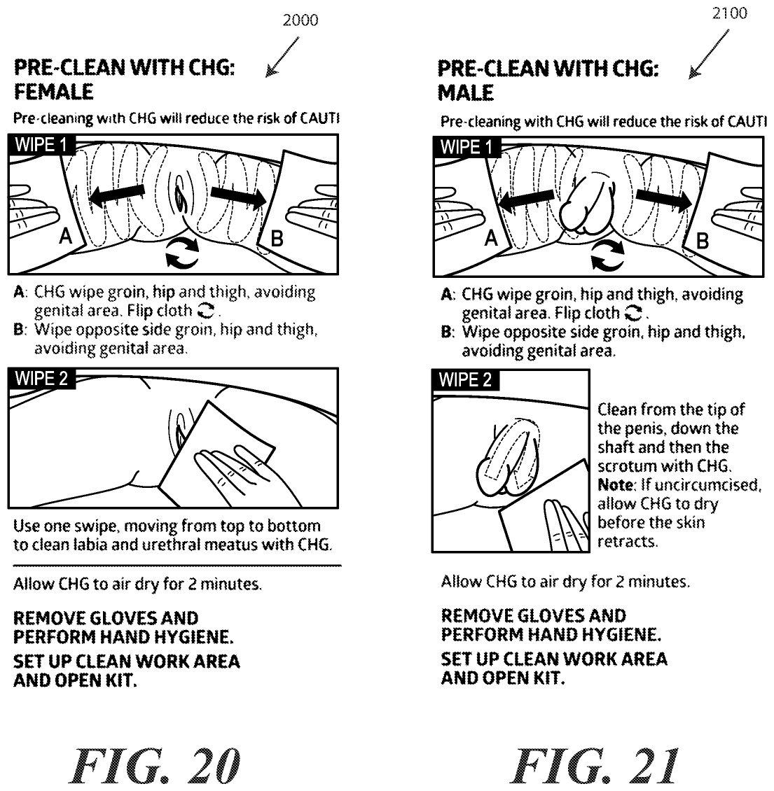

While the instructional banner 1800 of FIG. 18 illustrated generally how to prepare a patient for a Foley catheter insertion, sometimes additional preparation instructions will be required. Illustrating by example, in some applications, a patient will need to be pre-cleaned prior to having their perineal regions prepared for the Foley catheter insertion. Turning now to FIGS. 20-21, illustrated therein are two such instructional banners 2000,2100.

The instructional banner 2000 of FIG. 20 includes instructions for pre-cleaning a female patient. Similarly, the instructional banner of FIG. 21 includes instructions for pre-cleaning a male patient. These instructional banners 2000,2100 can include instructions for wiping the patient's legs in addition to cleaning the urinary outputs of each patient, as shown in these figures.

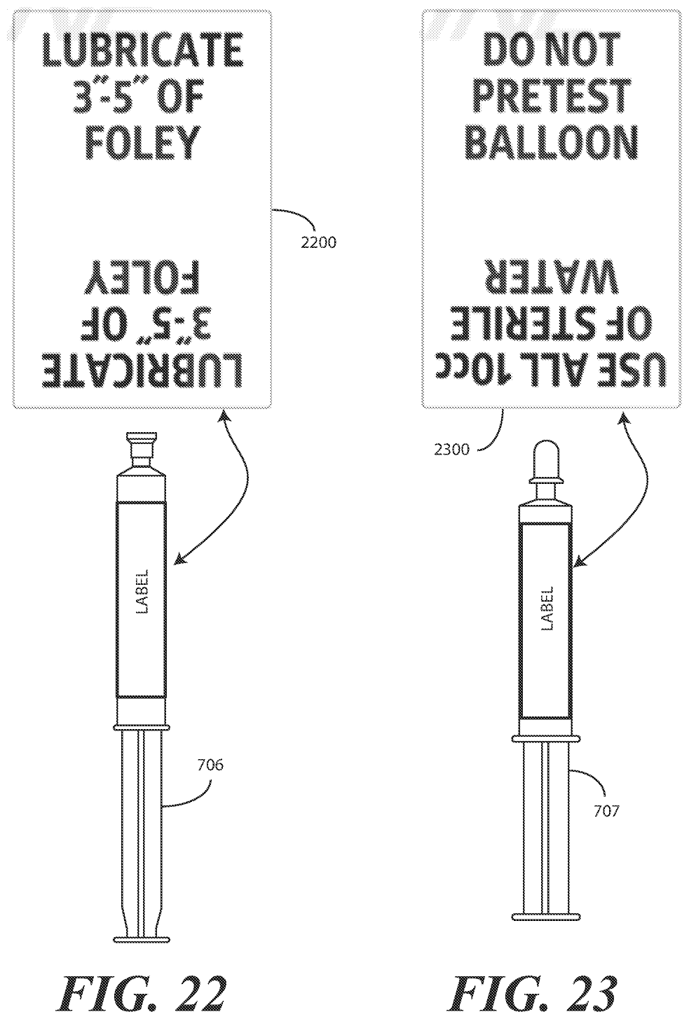

Turning now to FIG. 22, illustrated therein is one explanatory instructional banner 2200 suitable for attachment to a syringe 706 of lubricating jelly. In this illustrative embodiment, the instructional banner 2200 instructs a health care services provider to use the lubricant in the syringe 706 to lubricate 3-5 inches of the Foley catheter using the lubricating jelly application chamber defined by a compartment of the tray with which the syringe 706 is included as previously described. In one or more embodiments, these instructions can be disposed on the instructional banner both right side up and upside down, as shown in FIG. 22.

Turning now to FIG. 23, illustrated therein is one explanatory instructional banner 2300 suitable for attachment to a syringe 707 of sterile water. In this illustrative embodiment, the instructional banner 2300 instructs a health care services provider not to pre-test the inflation balloon of the Foley catheter, as this will make insertion of the same into a patient difficult or impossible. Additionally, the instructional banner 2300 instructs the health care services provider to use all the sterile water to inflate the balloon once the Foley catheter is applied to a patient. In one or more embodiments, the former is printed right side up while the latter is printed upside down, as shown in FIG. 23.

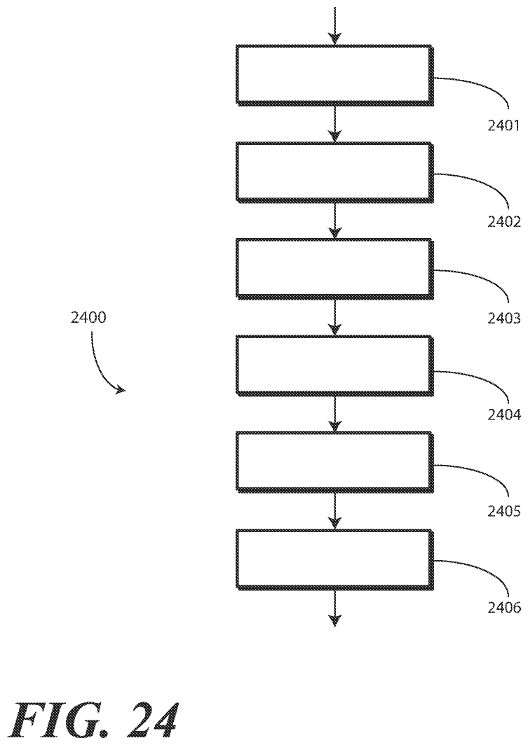

Turning now to FIG. 24, illustrated therein is one explanatory method 2400 for using the instructional banners shown in FIGS. 16-23. At step 2401, the instructional banners (2000,2100) of FIGS. 20-21 are attached to the back of a tray. At step 2402, the instructional banner (1700) of FIG. 17 is placed atop the package of liquid skin sanitizer and the rubber gloves included with the medical kit. At step 2403, the instructional banner (1600) of FIG. 16 is placed atop a patient aid when assembling a Foley catheter medical kit as described above with reference to FIGS. 8-15.

At step 2404, the instructional banners (1800,1900) of FIGS. 18-19 are attached to one or more layers of wrap material that are folded about a tray. At step 2405, the instructional banners (2200,2300) of FIGS. 23-24 are attached to a syringe of lubricating jelly and a syringe of sterile water, respectively. Steps 2406 then includes assembling a Foley catheter medical kit as described above with reference to FIGS. 8-15.

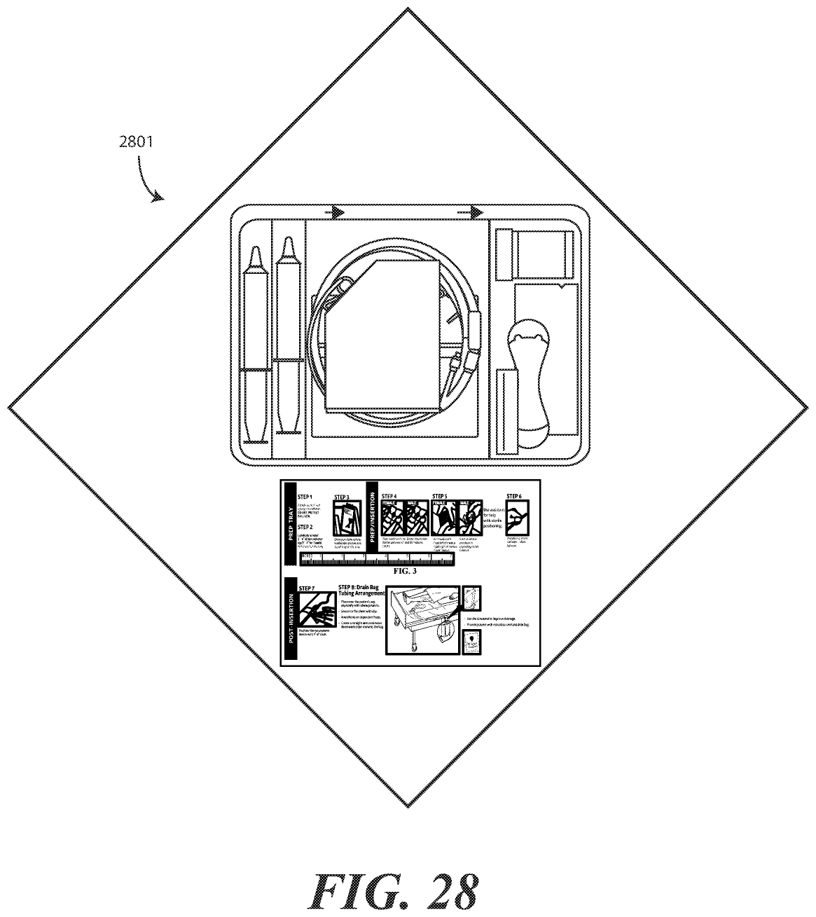

Accordingly, when assembling a medical kit using the method 2400 of FIG. 24, the method 2400 would include first include attaching the instructional banners (2000,2100) of FIGS. 20-21 are attached to the back of a tray in accordance with step 2401. The result of this step 2401 is shown in FIG. 25. The syringes 706,707 shown in FIG. 28 include the instructional banners (2200,2300) of FIGS. 23-24 attached thereto in accordance with step 2405.

Next, the method 2400 includes placing a Foley catheter assembly (700) within the first compartment (101) of the tray (100) at step 2406 as previously described. A folded drape (711) can optionally be placed atop the tray (100) at step 2406. The tray (100) is then placed upon one or more layers of wrap material (800) at step 2406. The one or more layers of wrap material (800) have the instructional banners (1800,1900) of FIGS. 18-19 attached thereto in accordance with step 2404. This partial medical kit 2801 is shown in FIG. 28. Unfolding the portion of the one or more layers of wrap material (800) to which the instructional banners (1800,1900) are attached from around the tray (100) reveals the instructional banners (1800,1900) in one or more embodiments.

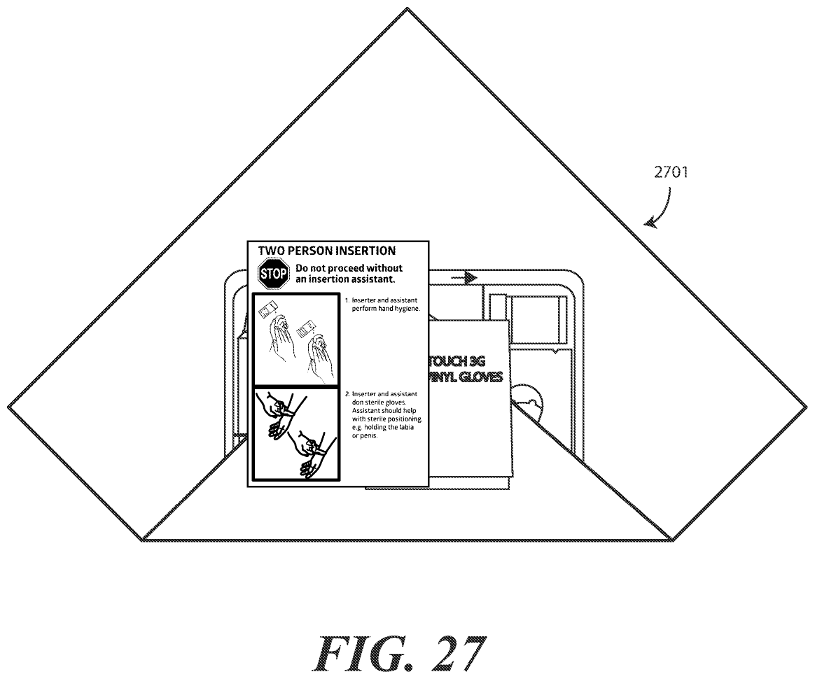

Returning to step 2406, at this step 2406 a first portion (901) of the one or more layers of wrap material (800) is folded about a first side (801) of the tray (100). Step 2406 can optionally include placing a package of skin sanitizer (709) and a package of rubber gloves (712) atop the first portion (901) of the wrap material (800). Step 2402 then includes placing the instructional banner (1700) of FIG. 17 atop the package of liquid skin sanitizer (709) and the rubber gloves (712) included with the medical kit. This partial medical kit 2701 is shown in FIG. 27.

Returning to step 2406, a second portion (1001) of the one or more layers of wrap material (800) is then folded about a second side (802) of the tray (100). In one or more embodiments, this second portion (1001) of the one or more layers of wrap material (800) coverthe package of liquid skin sanitizer (709), the package of rubber gloves (712), and the instructional banner (1700) of FIG. 17. In other embodiments, the second portion (1001) of the one or more layers of wrap material (800) may be folded so as to cover or partially cover these items at step 2406.

Step 2406 then includes folding a third portion (1101) of the one or more layers of wrap material (800) is folded about a third side (803) of the tray (100). Where the optional package of liquid skin sanitizer (709), package of rubber gloves (712), and the instructional banner (1700) of FIG. 17 are included, the third portion (1101) of the one or more layers of wrap material (800) may be folded so as to cover or partially cover these items. One or more towels (710) can optionally be placed atop the third portion (1101) of the one or more layers of wrap material (800) as well.

Step 2406 can then include placement of an additional layer of wrap material (1201) atop the partially assembled kit. The additional layer of wrap material (1201) can include a folded layer of CSR wrap measuring 17 by 17.5 inches. The additional layer of wrap material (1201) can be folded as a 4 by 2 matrix. As with the package of liquid skin sanitizer (709), the package of rubber gloves (712), and the instructional banner (1700) of FIG. 17, in one embodiment the additional layer of wrap material (1201) can be placed atop portions of the one or more layers of wrap material (800). In such an embodiment, the additional layer of wrap material (1201) can be held in place by way of subsequent folding steps, as the additional layer of wrap material (1201) is disposed along other folded portions of the one or more layers of wrap material (800) prior to folding a fourth portion of the one or more layers of wrap material about the fourth side (804) of the tray (100). One or more cleaning wipes (1202) can optionally be placed atop the additional layer of wrap material (1201) as well at step 2406.

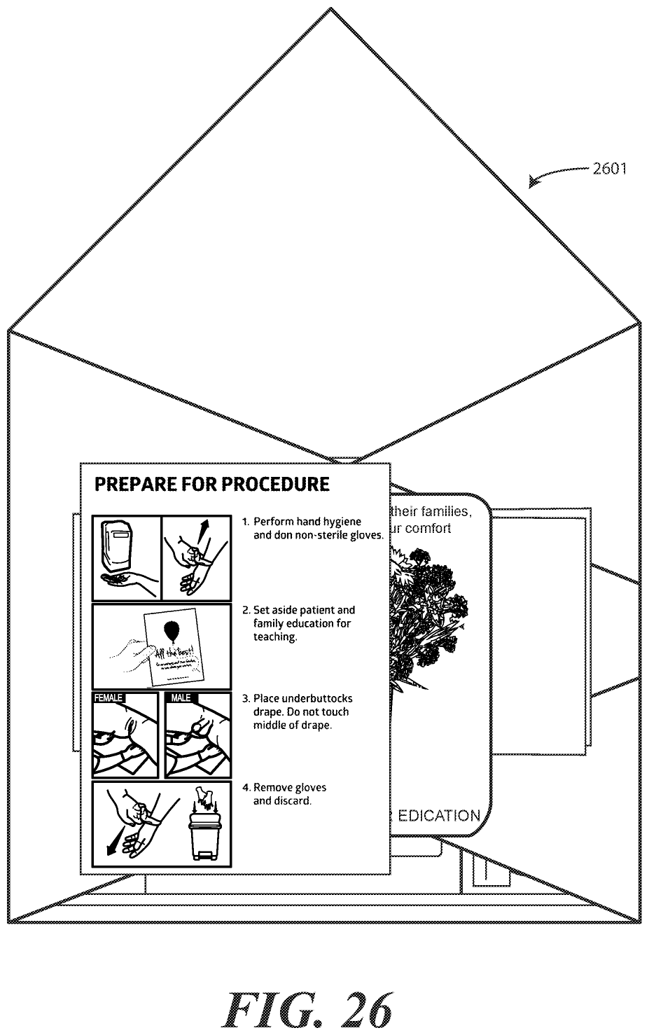

Step 2406 can then include placing a patient aid (1301) atop the portions of the one or more layers of wrap material (800) that have thus far been folded about the tray (100). Step 2403 can then include placing the instructional banner (1600) of FIG. 16 atop a patient aid (1301). This partial medical kit 2601 is shown in FIG. 26.

Step 2406 then includes folding a fourth portion (1401) of the one or more layers of wrap material (800) about a fourth side (804) of the tray (100) and then tucking at least one of the first portion (901), the second portion (1001), the third portion (1101), or the fourth portion (1401) of the one or more layers of wrap material (800) beneath at least another of the first portion (901), the second portion (1001), the third portion (1101), or the fourth portion (1401) of the layer of wrap material (800). Step 2406 can then include sealing wrapped Foley catheter assembly (1500) in a bag (1501). Prior to depositing the packaged Foley catheter assembly (1500) into the bag (1501), optional printed instructions (1502) can be attached to or disposed upon the packaged Foley catheter assembly (1500) as well as previously described. The assembly can be sealed in a sterile wrap such as a bag (1501), which may be thermally or otherwise sealed. Inclusion of a sterile wrap not only keeps the contents within the bag sterile, but also allows the printed various instructional banners to be sealed within the bag (1501) as well.

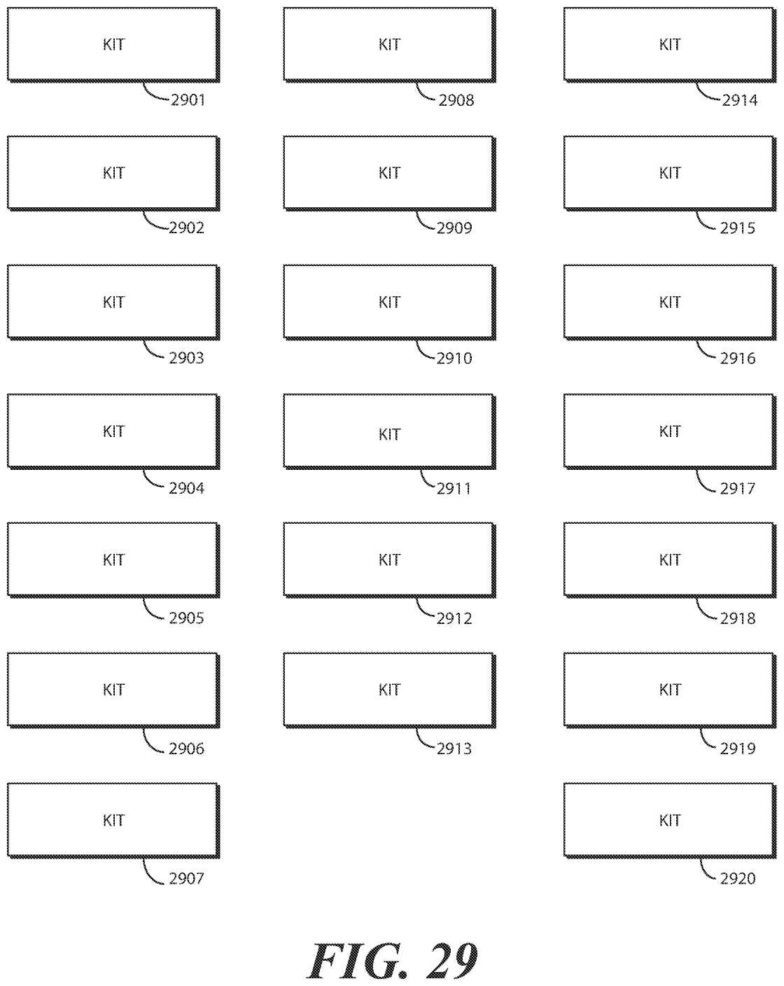

Turning now to FIG. 29, illustrated therein are various embodiments of the disclosure.

The embodiments of FIG. 29 are shown as labeled boxes in FIG. 29 due to the fact that the individual components of these embodiments have been illustrated in detail in FIGS. 1-28, which precede FIG. 29. Accordingly, their repeated illustration is no longer essential for a proper understanding of these embodiments. Thus, the embodiments are shown as labeled boxes.

At 2901, a medical procedure kit comprises a tray comprising a compartment separated by a wall from a lubricating jelly application chamber. At 2901, the kit comprises a container of lubricating jelly disposed within the lubricating jelly application chamber.

At 2901, the kit comprises a coiled tubing coupled between a Foley catheter and a drain bag, wherein the coiled tubing, the drain bag, and the Foley catheter are disposed within the compartment. At 2901, the kit comprises a first instructional banner attached to an underside of the compartment.

At 2902, the first instructional banner of 2901 depicts a pre-Foley catheter insertion cleaning process. At 2903, the medical procedure kit of 2901 comprises a second instructional banner attached to the underside of the compartment.

At 2904, the first instructional banner of 2903 comprises a female Foley catheter pre-cleaning instructional banner. At 2905, the second instructional banner of 2904 comprises a male Foley catheter pre-cleaning instructional banner.

At 2906, the medical procedure kit of 2901 further comprises an instructional banner attached to the container of lubricating jelly. At 2907, the medical procedure kit of 2906 further comprises a container of sterile water disposed within the lubricating jelly application chamber. At 2907, the container of sterile water has another instructional banner attached thereto.

At 2908, the medical procedure kit of 2901 further comprises one or more layers of wrap folded about the tray. At 2909, the medical procedure kit of 2908 further comprises an instructional banner attached to the one or more layers of wrap folded about the tray.

At 2910, unfolding a portion of the one or more layers of wrap material of 2909 from around the tray reveals the instructional banner. At 2911, the medical procedure kit of 2910 further comprises another instructional banner attached to the portion of the one or more layers of wrap material. At 2911, the instructional banner comprises a pre-insertion Foley catheter instructional banner and the other instructional banner comprises a post-insertion Foley catheter instructional banner.

At 2912, the medical procedure kit of 2908 further comprises a patient aid held within one or more folds of the one or more layers of wrap material. At 2913, the medical procedure kit of 2912 further comprises an instructional banner positioned atop the patient aid and within the one or more folds of the one or more layers of wrap material.

At 2914, a medical procedure kit comprises a single layer tray comprising a container of lubricating jelly, a container of water, and a coiled tubing coupling a Foley catheter to a drain bag. At 2914, each of the container of lubricating jelly, the container of water, the coiled tubing, the Foley catheter, and the drain bag are positioned within the single layer tray.

At 2914, the kit comprises a lubricating jelly application chamber defined by contours of the single layer tray. At 2914, the container of lubricating jelly is positioned in the lubricating jelly application chamber and the lubricating jelly application chamber is configured to receive lubricating jelly from the container of lubricating jelly, after the container of lubricating jelly is removed from the lubricating jelly application chamber, for lubricating at least a portion of the Foley catheter when the lubricating jelly is in the lubricating jelly application chamber.

At 2914, the kit comprises one or more layers of wrap material folded about the single layer tray. At 2914, at least one instructional banner is positioned within one or more folds of the one or more layers of wrap material.

At 2915, the at least one instructional banner of 2914 depicts a pre-Foley catheter insertion preparation process. At 2916, the at least one instructional banner of 2914 comprises a first instructional banner attached to the container of lubricating jelly. At 2916, the at least one instructional banner of 2915 comprises a second instructional banner attached to the container of water. At 2916, the at least one instructional banner of 2915 comprises at least a third instructional banner attached to the one or more layers of wrap material. At 2917, unfolding a portion of the one or more layers of wrap material of 2916 from around the tray reveals the at least a third instructional banner.

At 2918, a medical procedure kit comprises a single layer tray defining a wall separating a compartment from a lubricating jelly application chamber. At 2919, the kit comprises a container of lubricating jelly situated in the lubricating jelly application chamber.

At 2918, the kit comprises a coiled tubing coupled between a Foley catheter and a drain bag situated within the compartment. At 2918, the kit comprises one or more layers of wrap material folded about the single layer tray with a first instructional banner attached to a fold of the one or more layers of wrap material. At 2918, the lubricating jelly application chamber is configured to receive at least some lubricating jelly from the container of lubricating jelly for lubricating at least a portion of the Foley catheter with the at least some lubricating jelly when the at least some lubricating jelly is in the lubricating jelly application chamber.

At 2918, the first instructional banner of 2918 depicts a pre-Foley catheter insertion process. At 2920, the medical procedure kit of 2919 further comprises a second instructional banner held in place atop the single layer tray by one or more folds of the one or more layers of wrap material.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims.

Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A medical procedure kit, comprising:
a tray comprising a compartment separated by a wall from a lubricating jelly application chamber;
a container of lubricating jelly disposed within the lubricating jelly application chamber;
a coiled tubing coupled between a Foley catheter and a drain bag, wherein the coiled tubing, the drain bag, and the Foley catheter are disposed within the compartment; and
a first instructional banner attached to an underside of the compartment.

2. The medical procedure kit of claim 1, the first instructional banner depicting a pre-Foley catheter insertion cleaning process.

3. The medical procedure kit of claim 1, further comprising a second instructional banner attached to the underside of the compartment.

4. The medical procedure kit of claim 3, wherein the first instructional banner comprises a female Foley catheter pre-cleaning instructional banner.

5. The medical procedure kit of claim 4, wherein the second instructional banner comprises a male Foley catheter pre-cleaning instructional banner.

6. The medical procedure kit of claim 1, further comprising an instructional banner attached to the container of lubricating jelly.

7. The medical procedure kit of claim 6, further comprising a container of sterile water disposed within the lubricating jelly application chamber, wherein the container of sterile water has another instructional banner attached thereto.

8. The medical procedure kit of claim 1, further comprising one or more layers of wrap material folded about the tray.

9. The medical procedure kit of claim 8, further comprising an instructional banner attached to the one or more layers of wrap material folded about the tray.

10. The medical procedure kit of claim 9, wherein unfolding a portion of the one or more layers of wrap material from around the tray reveals the instructional banner.

11. The medical procedure kit of claim 10, further comprising another instructional banner attached to the portion of the one or more layers of wrap material, wherein the instructional banner comprises a pre-insertion Foley catheter instructional banner and the another instructional banner comprises a post-insertion Foley catheter instructional banner.

12. The medical procedure kit of claim 8, further comprising a patient aid held within one or more folds of the one or more layers of wrap material.

13. The medical procedure kit of claim 12, further comprising an instructional banner positioned atop the patient aid and within the one or more folds of the one or more layers of wrap material.

14. A medical procedure kit, comprising:
a single layer tray comprising a container of lubricating jelly, a container of water, and a coiled tubing coupling a Foley catheter to a drain bag, wherein each of the container of lubricating jelly, the container of water, the coiled tubing, the Foley catheter, and the drain bag are positioned within the single layer tray;
a lubricating jelly application chamber defined by contours of the single layer tray, wherein the container of lubricating jelly is positioned in the lubricating jelly application chamber and the lubricating jelly application chamber is configured to receive lubricating jelly from the container of lubricating jelly, after the container of lubricating jelly is removed from the lubricating jelly application chamber, for lubricating at least a portion of the Foley catheter when the lubricating jelly is in the lubricating jelly application chamber; and
one or more layers of wrap material folded about the single layer tray with at least one instructional banner positioned within one or more folds of the one or more layers of wrap material.

15. The medical procedure kit of claim 14, the at least one instructional banner depicting a pre-Foley catheter insertion preparation process.

16. The medical procedure kit of claim 14, the at least one instructional banner comprising:
a first instructional banner attached to the container of lubricating jelly;
a second instructional banner attached to the container of water; and
at least a third instructional banner attached to the one or more layers of wrap material.

17. The medical procedure kit of claim 16, wherein unfolding a portion of the one or more layers of wrap material from around the single layer tray reveals the at least a third instructional banner.

18. A medical procedure kit, comprising:
a single layer tray defining a wall separating a compartment from a lubricating jelly application chamber;
a container of lubricating jelly situated in the lubricating jelly application chamber;
a coiled tubing coupled between a Foley catheter and a drain bag situated within the compartment; and
one or more layers of wrap material folded about the single layer tray with a first instructional banner attached to a fold of the one or more layers of wrap material;
wherein the lubricating jelly application chamber is configured to receive at least some lubricating jelly from the container of lubricating jelly for lubricating at least a portion of the Foley catheter with the at least some lubricating jelly when the at least some lubricating jelly is in the lubricating jelly application chamber.

19. The medical procedure kit of claim 18, the first instructional banner depicting a pre-Foley catheter insertion process.

20. The medical procedure kit of claim 18, further comprising a second instructional banner held in place atop the single layer tray by one or more folds of the one or more layers of wrap material.
